(19) United States
(12) Patent Application Publication
(10) Pub. No.: US 2013/0023480 A1
(43) Pub. Date: Jan. 24, 2013

(54) Purified Bowman-Birk Protease Inhibitor Proteins Isolated from a Soy Processing Sttream

(71) Applicants: Charles S. Schasteen, University City, MO (US); Darrell Marsh, High Ridge, MO (US); Karsten Keller, Hockessin, MO (US); Marlene Mekel, High Ridge, MO (US); Jennifer Wu, St. Charles, MO (US); Barry Tulk, Creve Coeur, MO (US)

(72) Inventors: Charles S. Schasteen, University City, MO (US); Darrell Marsh, High Ridge, MO (US); Karsten Keller, Hockessin, MO (US); Marlene Mekel, High Ridge, MO (US); Jennifer Wu, St. Charles, MO (US); Barry Tulk, Creve Coeur, MO (US)

(73) Assignee: SOLAE, LLC, St. Louis, MO (US)

(21) Appl. No.: 13/519,832

(22) PCT Filed: Dec. 30, 2010
(86) PCT No.: PCT/US10/62553

§ 371 Date: Jun. 28, 2012

Related U.S. Application Data
(60) Provisional application No. 61/291,312, filed on Dec. 30, 2009.

(51) Int. Cl. C07K 14/81; A61P 35/00; A61P 37/00; A61P 17/00; A61K 38/56; A61P 21/00

(52) U.S. Cl. 514/18.6; 530/300; 530/324; 514/20.3; 514/19.3

(57) ABSTRACT

Purified Bowman-Birk protease inhibitor (BBI) proteins recovered from a soy processing stream are disclosed, as well as a process for recovering and isolating purified BBI proteins from a soy processing stream.

### CROSS REFERENCE TO RELATED APPLICATIONS

This application claims priority from U.S. Provisional Application Ser. No. 61/291,312 filed on Dec. 30, 2009, which is hereby incorporated by reference in its entirety.

### BACKGROUND OF THE INVENTION

The protease inhibitor, Bowman-Birk protease inhibitor (BBI), is a low molecular weight protein (7-8 kDa) double-headed inhibitor of trypsin and chymotrypsin isolated from soybeans. It was first discovered over sixty years ago (Bowman, Proc. Soc. Exptl. Med., 1946, 63, 574; and subsequently further characterized by Birk, Y. Biochim. Biophys. Acta, 1961, 54, 378-381; and Birk. Y. et al., Biochemical Preparations, 1968, Vol. 12, 25-29) and has attracted renewed interest from the scientific research community since the discovery of its potent anticarcinogenic effects in several experimental systems.

In addition to inhibiting trypsin and chymotrypsin, BBI also has the ability to inhibit the activity of other proteases, such as cathepsin G, elastase, and chymase (Birk Y., Int J Pept Protein Res, 1985, 25: 113-131; Larionova et al., Biokhimiya, 1993, 58: 1437-1444; and Ware et al., Archives of Biochemistry and Biophysics 1997, 344: 133-138, each of which is incorporated herein by reference). The BBI protein consists of approximately 65-77 amino acid residues and approximately seven disulfide bridges. BBI is a protein characterized by its high concentration (˜20 wt. %) of the amino acid cysteine, high aqueous solubility, resistance to heat denaturation and having the capacity to inhibit trypsin and chymotrypsin at independent inhibitory sites.

It is well-known that both crude and purified BBI prevent or reduce various types of induced malignant transformation of cells in culture and experimental animals (Kennedy, A. R., The Bowman-Birk Inhibitor from soybeans as an anticarcinogenic agent, Am J of Clinical Nutr, 1998: 68, 1406S-1412S). See, also, for example: (1) Kennedy, A. R. Chemopreventive agents: protease inhibitors. Pharmacology & Therapeutics 78: 167-209, 1998; (2) Kennedy, A. R. Overview: Anticarcinogenic activity of protease inhibitors. In: Protease Inhibitors as Cancer Chemopreventive Agents; (3) Troll, W., Kennedy, A. R., Eds.; Plenum Publishing Corporation New York, 9-64, 1993; (4) Kennedy, A. R., Szuhaj, B. F., Newberne, P. M., Billings, P. C. Preparation and production of a cancer chemopreventive agent, Bowman-Birk Inhibitor Concentrate. Nutr. Cancer 19: 281-302, 1993; (5) Kennedy, A. R. Prevention of carcinogenesis by protease inhibitors. Cancer Res. (suppl.). 54: 1999s-2005s, 1994; (6) Kennedy, A. R. In vitro studies of anticarcinogenic protease inhibitors. In: Protease Inhibitors as Cancer Chemopreventive Agents; Troll, W., Kennedy, A. R., Eds.; Plenum Publishing Corporation New York, 65-91, 1993 (7) Kennedy, A. R., The Status of Human Trials Utilizing Bowman-Birk Inhibitor Concentrate from Soybeans. In: Soy in Health and Disease Prevention, edited by Michihiro Sugano, CRC Press LLC, Boca Raton, Fla., Chapter 12, pp. 207-223, 2005; (8) Kennedy, A. R. Status of current human trials utilizing Bowman Birk Inhibitor Concentrate. Proceedings of a Symposium, “Soy & Health 2006; Dietetic Applications-Dietetic Applications”, held on Oct. 12 and 13, 2006, Dusseldorf, Germany (in press); (9) Bartsch and Gerhauser, Molecular Mechanisms of Cancer Induction and Chemoprevention. In: Chemoprevention of Cancer and DNA Damage by Dietary Factors, edited by Siegfried Knasmuller, Ian Johnson, David DeMarini and Clarissa Gerhauser, Wiley VCH Verlag, GmbH & Co., KGaA, Weinheim, 2009.

A soybean extract enriched in BBI, commonly referred to as Bowman-Birk Inhibitor Concentrate (BBIC) has achieved Investigational New Drug (IND) Status with the Food and Drug Administration (FDA) in April of 1992. BBIC has been shown to exhibit inhibitory activity against the malignant transformation of cells under certain conditions and its administration has been shown to affect various forms of cancer. See, for example, U.S. Pat. No. 7,404,973. By way of further example, animals maintained on 1.0% dietary BBIC for their entire life have been shown to have had no growth abnormalities and were found to have a significantly extended life span (Kennedy et al. Nutr Cancer, 1993, 19: 281-302).

BBIC has also been shown to have activity in treatment of oral cancer, muscular dystrophy, prevention of muscle wasting, anti-inflammatory activity, radioprotective activity in animal models and human clinical trials. (See, for example, Kennedy, A. R., Soy and Health and Disease Prevention, 2005 and Sweeney et al. U.S. Patent Publication No. US 2008/0300179 A1). BBIC has also been shown to inhibit proteolytic activity in lung, kidney and liver tissue following intra-peritoneal injections in mice (Oreffo et al., Toxicology, 1991, 69: 165-176). BBIC has also been shown to ameliorate the effects of neuromuscular diseases (U.S. Patent Application Publication No. 20080300179, Morris et al., J Appl Physiol. 2005 November; 99(5):1719-27, Arbogast et al., J Appl Physiol. 2007 March; 102(3):956-64).

It is currently believed that the BBI products of the present disclosure are suitable for incorporation in a variety of compositions suitable for any or all of these purposes.

In view of the possibility that a BBI product may provide a potential remedy for prevention and amelioration of carcinogenesis, attempts have been made to prepare pure and sundry BBIC preparations as potential therapeutic medicaments for diverse cancer conditions by various methods (U.S. Pat. No. 5,217,717; a review of the relevant literature is provided by Kennedy et al. in U.S. Pat. No. 5,338,547, which is hereby incorporated in its entirety). U.S. Pat. No. 4,793,996, also Kennedy et al., discloses a process of treating soybeans with acetone, followed by ethanol extraction and acetone precipitation for obtaining BBIC. Kennedy et al. discovered that by treating the soybeans with acetone prior to the ethanol extraction step taught by Perlmann et al., Methods in Enzymology, 19: 860-861 (1970), the resulting BBIC was more effective in inhibiting the malignant transformation of cells.

The above mentioned U.S. Pat. No. 5,338,547, discloses a method for suppressing and inhibiting carcinogenesis with highly active BBI concentrate (BBIC) products wherein the level of biological activity is measured by chymotrypsin inhibitor content. These BBI concentrate products are made from acidic soybean solubles obtained from defatted soybean flour or flakes which were extracted with aqueous acid at pH 4 to 5, and from which the insolubles were removed by centrifugation. The soybean solubles were subjected to ultrafiltration to produce a crude BBI concentrate, which was diluted and spray dried to produce the final dried BBI concentrate product. In a preferred process embodiment disclosed in this patent, the crude BBI concentrate was treated with acetone to produce a BBI concentrate precipitate which is air dried, ground, reslurried with water, filtered and then lyophilized or spray dried to produce the final BBI concentrate product. This product was stated to be an improved inhibitor of carcinogenesis. Kennedy et al. also mention that the BBI concentrate product can be further purified, by a method described by Odani et al. (J. Biochem. 1973, 74, 857), which method involves fragmenting the BBIC product into two separated fragments, one fragment having the trypsin inhibitory site and the other fragment having the chymotrypsin inhibitory site. The inhibiting activity of the fraction having the chymotrypsin inhibitory site was, however, severely impaired.

Current methods known in the art for obtaining purified BBI proteins suffer from lower purity levels due to the contamination of the BBI with Kunitz Trypsin Inhibitor (KTI) proteins. Depending on the isolation method used, endotoxin levels can also be an issue. Current methods use whole soybean as the starting material, which may then be defatted by various means. In contrast, the processes of the present invention use defatted soy white flake as the starting material. As a result, the prior art has not described a BBI product having high purity levels, and, in particular, the prior art has not described a BBI product having high purity levels obtained from soybean. In addition, it is noted that BBI was identified by Bowman in the 1940s and further characterized by Birk in the 1960s (Bowman D. E., Proc. Soc. Exp. Biol. Med., 63: 547-550, 1946; Birk, Y. Biochim. Biophys. Acta, 1961, 54, 378-381; and Birk. Y. et al., Biochemical Preparations, 1968, Vol. 12, 25-29). However, there is a void of any BBI product having purity levels as described herein in the literature or commercially.

Thus, there is a need for compositions and methods suitable for the production of high purity BBI products and variants. Accordingly, the present invention describes BBI products and novel BBI protein isoforms that have been recovered in high purity through methods disclosed herein.

### SUMMARY OF THE INVENTION

The present invention generally provides novel Bowman-Birk protease inhibitor (BBI) products that have been isolated and recovered from a soy processing stream, including soy whey streams and soy molasses streams, and that exhibit certain desirable characteristics.

The resultant purified BBI product of the present invention consists of a polypeptide having an amino acid sequence at least 90% identical to SEQ ID NO: 1-6; and further have a BBI purity represented by a total BBI protein concentration of at least about 90 wt. %. A BBI purity represented by a total BBI protein concentration of at least about 90 wt. % means that the BBI product of the present invention is comprised of at least about 90 wt. % BBI proteins, while the remaining 10 wt. % of the BBI product of the present invention is comprised of impurities. The impurities may include other proteins (such as storage proteins or acid soluble proteins, depending on the source of the soy whey protein), lipids, microorganisms, minerals, and sugars. In addition, the purified BBI product further exhibits (i) a total protein content of at least about 95% on a dry weight basis; (ii) a trypsin inhibitor activity of at least about 1200 trypsin inhibition units/g protein; (iii) a chymotrypsin inhibitor activity of at least about 1600 chymotrypsin inhibition units/g protein; and/or, (iv) a total endotoxin content of no more than about 5 endotoxin units (EU)/g protein. The amount of BBI product isolated by the processes of the present invention may be as small as a gram (lab scale isolation) or may be several metric tons (industrial or large scale isolation).

In addition to having high purity, the novel BBI proteins of the present invention remain effective for chymotrypsin and trypsin inhibition, exhibiting a ratio of chymotrypsin inhibitor activity to trypsin inhibitor activity of at least about 1:1, at least about 10:1, at least about 25:1, at least about 50:1, at least about 75:1, at least about 90:1, at least about 95:1, or at least about 99:1.

In another aspect, the BBI product comprising BBI proteins of the present invention results in one or more endotoxins, wherein the total endotoxin content is from about 0.1 to about 5.0 EU/g protein, from about 0.1 to about 2.5 EU/g protein, from about 0.1 to about 1.5 EU/g protein, from about 0.1 to about 1.0 EU/g protein, or from about 0.1 to about 0.5 EU/g protein.

The present disclosure is directed to a BBI product of previously unachieved levels of purity, which product is recovered from a soy processing stream. These isolated proteins are ultimately suitable for use in a variety of pharmaceutical applications and personal care products. Thus, in addition to the economic benefits over conventional BBI isolation and purification, the methods of the present disclosure likewise represent an advance in the art based on the nature of the BBI products provided by the present processes.

### BRIEF DESCRIPTION OF THE DRAWINGS

FIG. 1A is a schematic flow sheet depicting Steps 0 through 4 in a process for recovery of a purified soy whey protein from a processing stream.

FIG. 1B is a schematic flow sheet depicting Steps 5, 6, 14, 15, 16, and 17 in a process for recovery of a purified soy whey protein from a processing stream.

FIG. 1C is a schematic flow sheet depicting Steps 7 through 13 in a process for recovery of a purified soy whey protein from a processing stream.

FIG. 2 is a schematic flow sheet depicting a membrane based process for recovery of BBI proteins from a soy whey stream.

FIG. 3 depicts a sodium dodecyl sulfate polyacrylamide gel electrophoresis (SDS-PAGE) depicting various retentates and permeates generated during BBI purification according to the invention, including the resultant BBI product.

FIG. 4 illustrates the MALDI-TOF mass spectrometry data for certain of the novel BBI protein sequences isolated by the process of the present invention.

FIG. 5 depicts the BBI proteins of the present invention following two-dimensional polyacrylamide gel electrophoresis (2D-PAGE).

FIG. 6 depicts the results of 2D-PAGE analysis of a BBI product commercially sold.

FIG. 7 depicts the primary structure of BBI from soybean as known in the art according to Odani and Ikenaka.

FIG. 8 depicts novel BBI protein isoforms.

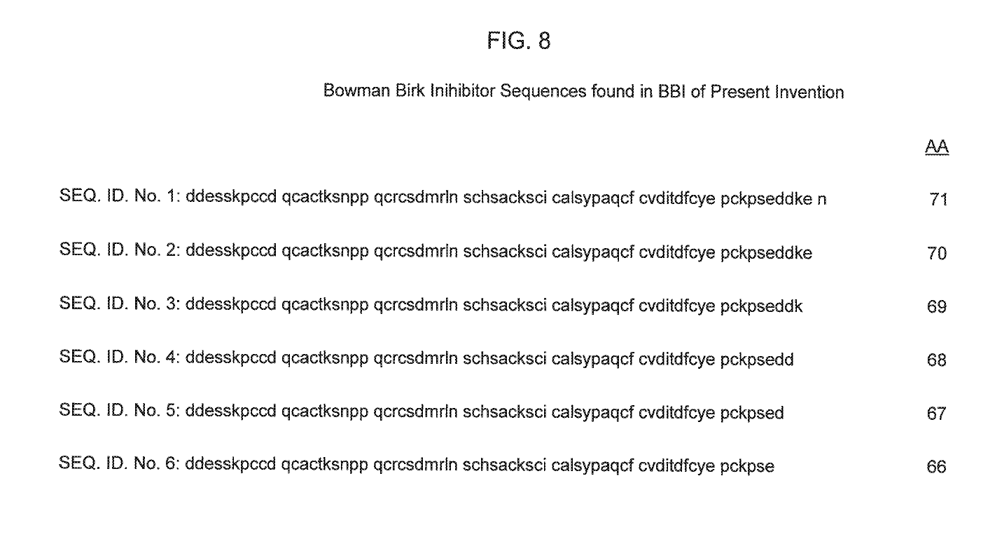

### DETAILED DESCRIPTION OF THE INVENTION

Described herein are novel BBI protein isoforms exhibiting desirable characteristics, including previously unachieved levels of purity, which BBI protein isoforms have been recovered from a soy processing stream. More particularly, as further described herein, the purified BBI protein isoforms are recovered from a concentrated aqueous soy whey stream generated in the manufacture of soy protein isolate. Generally, the processes of the present invention comprise a number of operations (i.e. membrane separation (or filtration) and chromatographic separation) selected and designed to provide isolation of the purified BBI proteins.

As detailed elsewhere herein, a highly purified fraction of BBI proteins can be recovered from aqueous soy whey following concentration. Recovery of targeted BBI proteins in high purity is improved by removal of other major components of the whey stream (e.g. minerals, sugars, and storage proteins, i.e. glycinin and β-conglycinin) that detract from purity by diluents, while likewise improving BBI protein concentration by purifying the BBI protein fraction through removal of components that are antagonists to the proteins and/or have deleterious effects (e.g. endotoxins).

Generally, several separation steps will be performed sequentially in order to recover the BBI proteins at the desired level of purity and to concentrate the process stream. For example, a purified fraction is typically first prepared by removal of one or more impurities (e.g. microorganisms or minerals), followed by removal of additional impurities including one or more soy storage proteins, followed by removal of one or more soy whey proteins (including, for example, KTI and other non-BBI proteins or peptides), and/or followed by removal of any remaining impurities, including sugars, from the soy whey. The particular filtration or separation step will depend on the type of component to be removed. For example, components having a smaller size will be removed through use of a separation membrane with a small pore size. As a further example, other components may be best suited for removal through an ion exchange column or by reverse osmosis. Removal of storage proteins, sugars, minerals, and other impurities yields fractions that are enriched in the desired BBI proteins and free of impurities that may be antagonists or toxins, or may otherwise have a deleterious effect.

### A. Bowman-Birk Protease Inhibitors

As discussed herein, soy processing streams, which include for example, soy whey stream and soy molasses stream, contain a significant amount of Bowman-Birk protease inhibitor (BBI). This protease inhibitor is known to at least inhibit trypsin, chymotrypsin and potentially a variety of other key proteases, such as cathepsin G, elastase, and chymase that regulate a range of key metabolic functions.

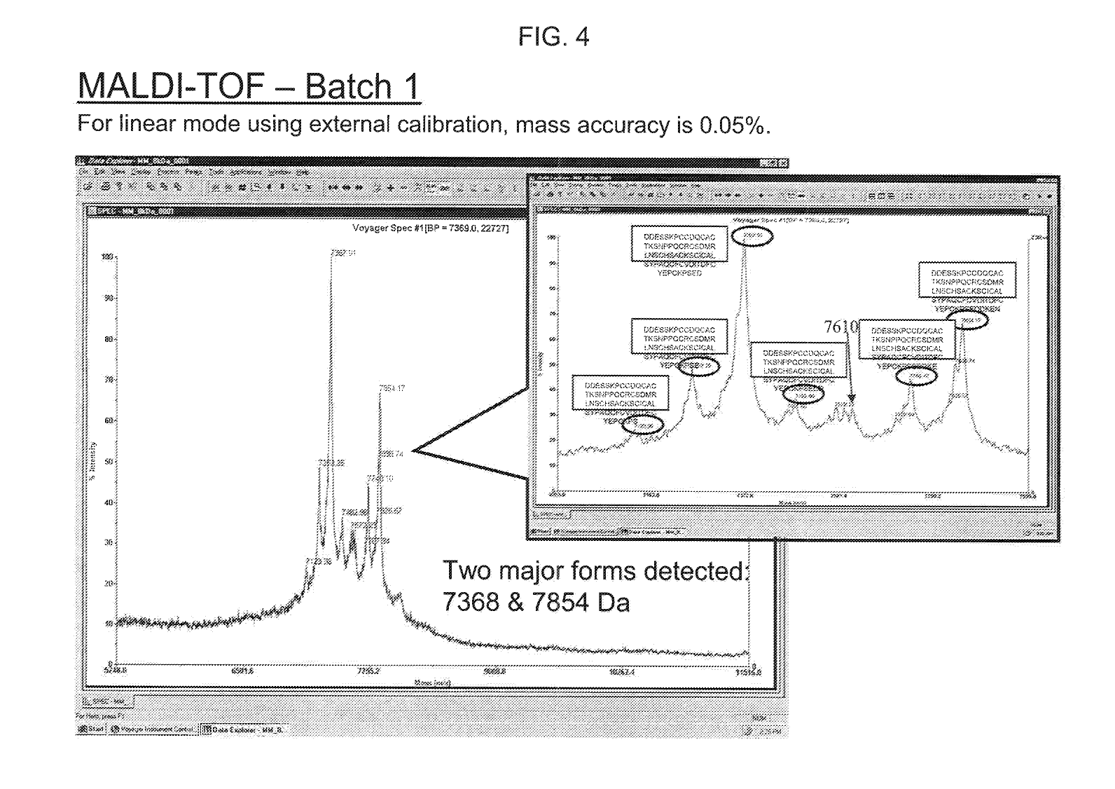

The BBI proteins isolated in accordance with the present embodiment may comprise a polypeptide having an amino acid sequence at least 50%, 60%, 65%, 70%, 75%, 80%, 85%, 90%, 95%, 99%, or even 100% identical to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof. FIG. 4 depicts the mass spectrometry data results of the novel BBI protein isoforms isolated by the present invention. In one embodiment, the BBI protein may comprise an amino acid sequence at least 70% identical to one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, and combinations thereof, more preferably at least 80% identical to one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, and combinations thereof, even more preferably at least 90% identical to one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, and combinations thereof, and most preferably at least 95% identical to one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

In another aspect of the present embodiment, the amino acid sequence is at least 50%, 60%, 65%, 70%, 75%, 80%, 85%, 90%, 95%, 99%, or even 100% identical to SEQ ID NO: 1.

In another aspect of the present embodiment, the amino acid sequence is at least 50%, 60%, 65%, 70%, 75%, 80%, 85%, 90%, 95%, 99%, or even 100% identical to SEQ ID NO: 2.

In another aspect of the present embodiment, the amino acid sequence is at least 50%, 60%, 65%, 70%, 75%, 80%, 85%, 90%, 95%, 99%, or even 100% identical to SEQ ID NO: 3.

In another aspect of the present embodiment, the amino acid sequence is at least 50%, 60%, 65%, 70%, 75%, 80%, 85%, 90%, 95%, 99%, or even 100% identical to SEQ ID NO: 4.

In another aspect of the present embodiment, the amino acid sequence is at least 50%, 60%, 65%, 70%, 75%, 80%, 85%, 90%, 95%, 99%, or even 100% identical to SEQ ID NO: 5.

In another aspect of the present embodiment, the amino acid sequence is at least 50%, 60%, 65%, 70%, 75%, 80%, 85%, 90%, 95%, 99%, or even 100% identical to SEQ ID NO: 6.

In certain aspects of the invention, sequence identity between two amino acid sequences is determined by comparing the amino acid sequences. In other aspects of the invention, sequence identity can be determined by comparing the amino acid sequences and its conserved amino acid substitutes. In other aspects of the invention, a protein of the invention can have one or more conservative substitutions. In other aspects of the invention, a protein of the invention can have one or more non-conservative substitutions.

Naturally occurring amino acids include, for example, alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V).

Conservative and non-conservative amino acid substitutions are known to those of ordinary skill in the art, for example, substituting an acidic amino acid for another acid amino acid may be considered a conservative substitution whereas substituting a basic amino acid for an acidic amino acid may be considered a non-conservative substitution; similarly, substituting a polar amino acid for another polar amino acid may be considered a conservative substitution whereas substituting a nonpolar amino acid for a polar amino acid may be considered a non-conservative substitution. Amino acids are generally grouped into the following categories (which can be used as a guide for determining whether a substitution is conservative or non-conservative): (1) polar/hydrophilic: N, Q, S, T, K, R, H, D, E, C, and Y; (2) non-polar/hydrophobic: G, A, L, V, I, P, F, W, and M; (3) acidic: D, E, and C; (4) basic: K, R, and H; (5) aromatic: F, W, Y, and H; and (6) aliphatic: G, A, L, V, I, and P.

In certain aspects of the invention wherein one or more amino acid sequences are not identical to SEQ ID NO: 1, SEQ ID NO: 2, SEQ ID NO: 3, SEQ ID NO: 4, SEQ ID NO: 5, or SEQ ID NO: 6, such one or more amino acid sequences also function as a BBI protein, which are known to inhibit both chymotrypsin and trypsin activity. Methods for ascertaining these functions are described herein and are known to one of ordinary skill in the art.

In other aspects of the invention wherein a composition comprises one or more amino acid sequences that are not identical to SEQ ID NO: 1, SEQ ID NO: 2, SEQ ID NO: 3, SEQ ID NO: 4, SEQ ID NO: 5, or SEQ ID NO: 6, such one or more amino acid sequences also function as a BBI protein, which are known to inhibit both chymotrypsin and trypsin. Methods for ascertaining these functions are described herein and are known to one of ordinary skill in the art.

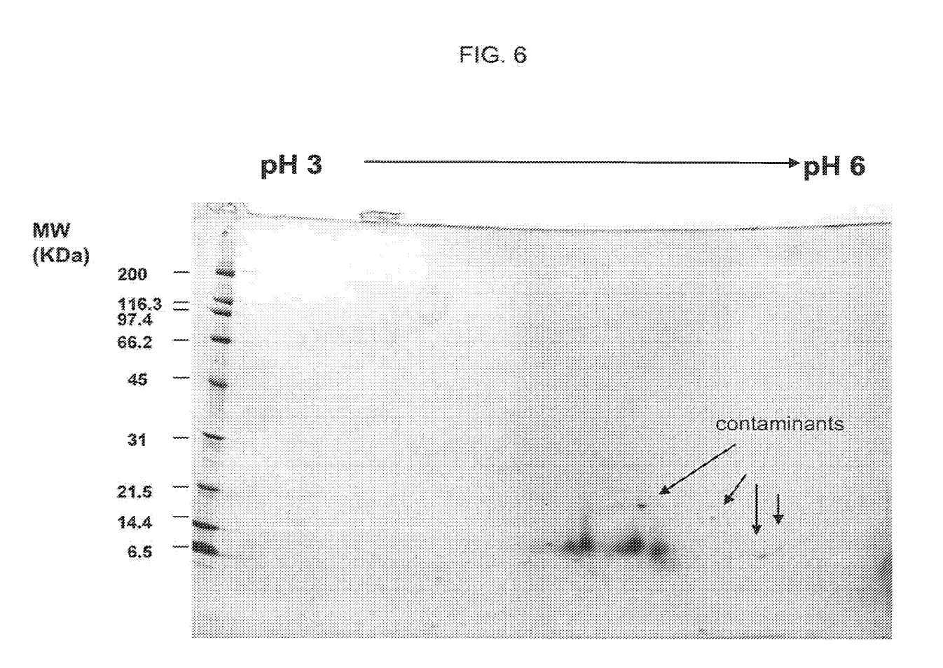

BBI proteins are comprised of approximately 65 to 77 amino acid residues and approximately seven disulfide bridges. The primary structure of a BBI protein has been known since 1972 (Odani S, and T. Ikenaka, J. Biochem. 1973 74:697 1972) and is as set forth in FIG. 7.

It is currently believed that the purity of the BBI products of the present disclosure represent previously unachieved levels of purity as compared to other BBI products. The purity of the BBI fraction is a function of total BBI protein concentration, specific activity (as measured by chymotrypsin inhibitor units/g protein), and the absence of components that function as antagonists for BBI, toxins, or other components that have deleterious effect beyond merely diluting the efficiency per unit quantity of the BBI. Generally, the total BBI protein concentration of BBI products of the present disclosure is at least about 70 wt. %, or at least about 80 wt. %. Typically, the total BBI protein concentration of the BBI products of the present disclosure is at least about 90 wt. %, at least about 91 wt. %, at least about 92 wt. %, at least about 93 wt. %, at least about 94 wt. %, at least about 95 wt. %, at least about 96 wt. %, at least about 97 wt. %, at least about 98 wt. %, and at least about 99 wt. %.

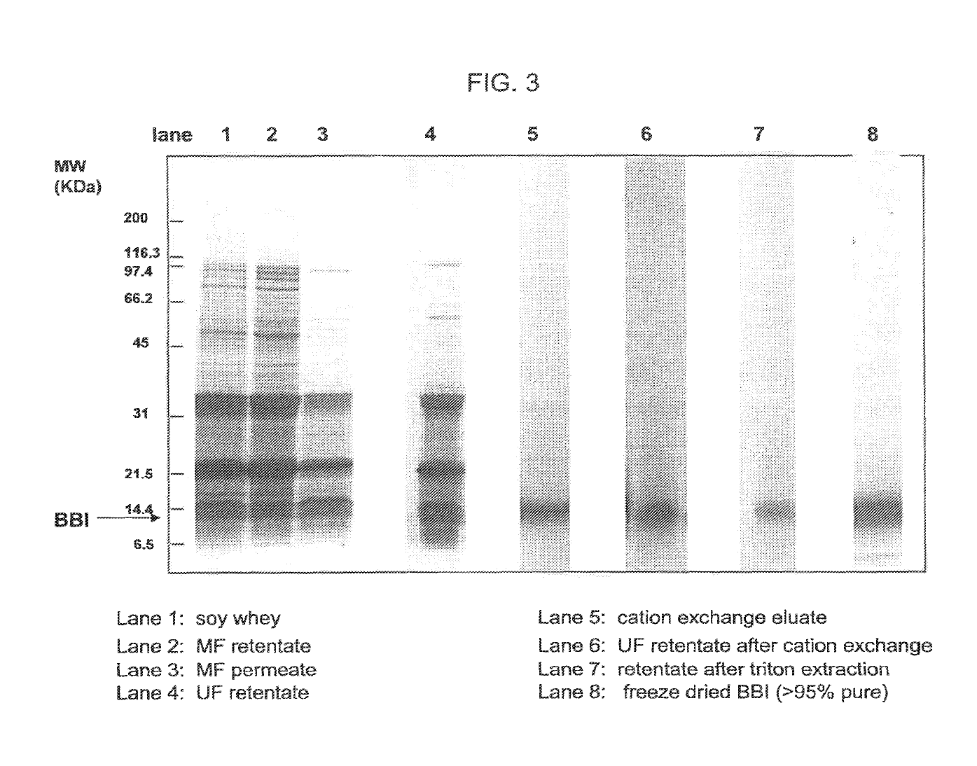

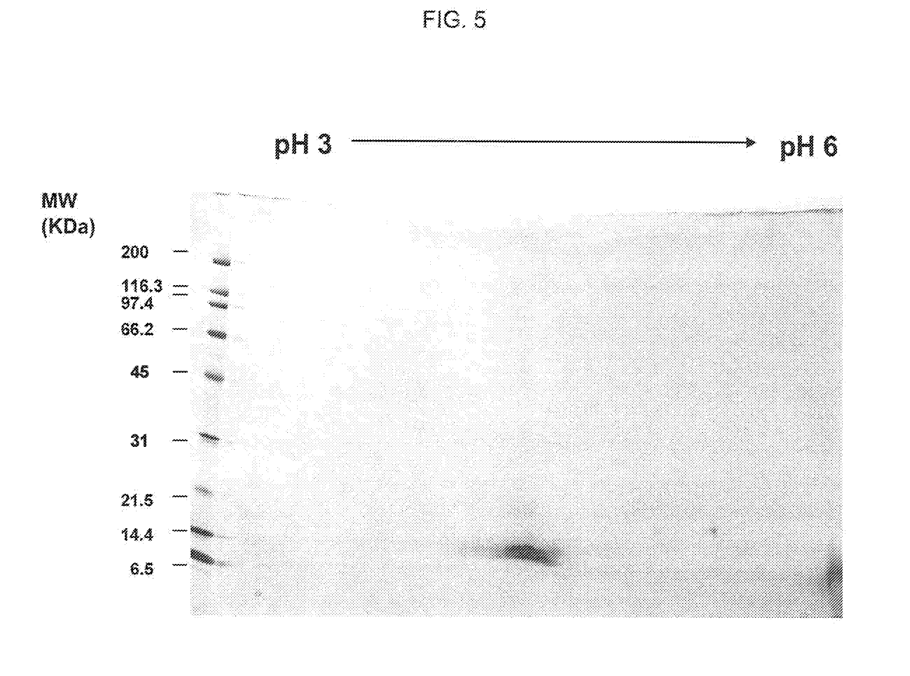

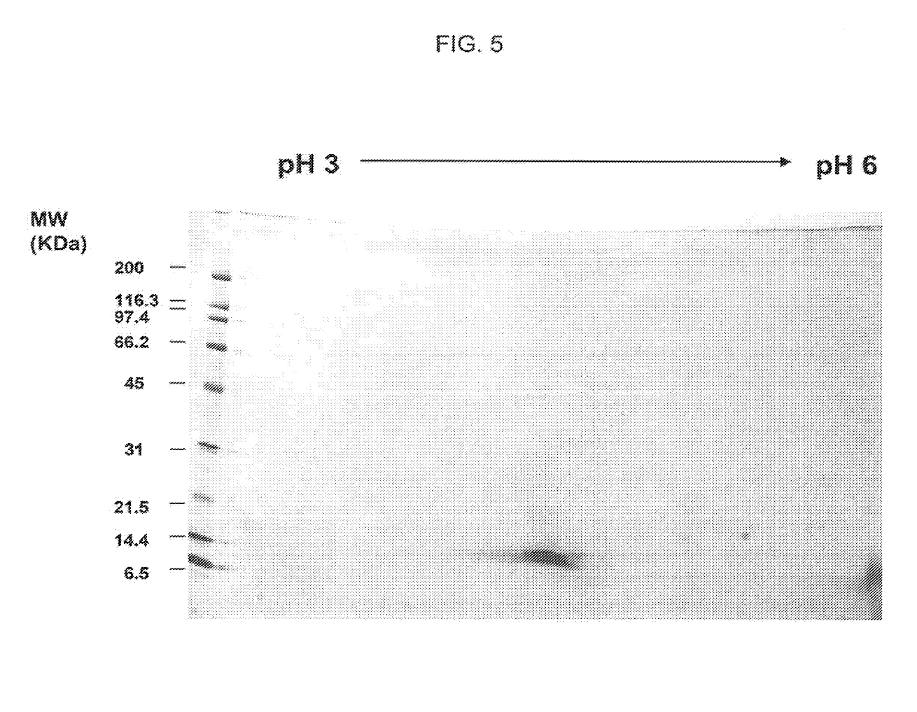

A “pure” monomeric protein will yield a single band after electrophoresis on a one or two-dimensional SDS-PAGE gel, will elute from a gel filtration, high performance liquid chromatography (HPLC), or ion exchange column as a single symmetrical absorbance peak, will yield a single set of mass spectrometric, nuclear magnetic resonance (NMR), or W absorbance spectral signals, and where appropriate, will be free of contaminating enzyme activities. Since absolute purity can never be established, a simple criterion of purity is used routinely, namely, the inability to detect more than a single band of protein after SDS-PAGE. (See Mohan, Determination of purity and yield. Methods in Molecular Biology, 11, 307-323 (1992)). FIG. 3 depicts the BBI proteins of the present invention following one-dimensional gel electrophoresis. FIG. 4 depicts the BBI proteins of the present invention following two-dimensional gel electrophoresis (2D-PAGE). As FIGS. 3 and 5 illustrate, the BBI proteins of the present invention showed as a single band between the molecular weight standards of 6.5 kDa and 14.4 kDa and with different isoelectric points. The presence of only a single band indicates the lack of contaminants in the product. In comparison, FIG. 6 depicts the results of 2D-PAGE analysis of a BBI product commercially sold by Sigma Aldrich, St. Louis, Mo. (product no. T9777). In FIG. 6, it is contrastingly apparent that while the sample BBI proteins were found between the same molecular weight standards as the BBI proteins in FIG. 5, they did not appear as a single band. This indicates that more contaminants, including residual Kunitz trypsin inhibitor proteins as well as non-protein components, were present in the Sigma BBI sample than in the BBI proteins of the present invention.

Along with BBI purity, the total protein content of the BBI products of the present disclosure is advantageous and/or represents an advance over the art. BBI protein content of products of the present disclosure may be determined by conventional methods known in the art including, for example, the Lowry method described in Ohnishi, S. T., and Barr, J. K., A simplified method of quantitating proteins using the biuret and phenol reagents. Anal. Biochem., 86, 193 (1978). Generally, the total protein content of the BBI products of the present disclosure is at least about 60 wt. % (on a dry weight basis), at least about 70 wt. %, at least about 80 wt. %, or at least about 85 wt. %. Typically, the total protein content of BBI products of the present disclosure is at least about 90 wt. %, at least about 91 wt. %, at least about 92 wt. %, at least about 93 wt. %, at least about 94 wt. %, at least about 95 wt. %, at least about 96 wt. %, at least about 97 wt. %, at least about 98 wt. % and at least about 99 wt. %.

Various applications for which the BBI products are currently believed to be suitable require relatively low endotoxin content. For example, various therapeutic applications require that the BBI product satisfy the applicable regulations for pharmaceutical-grade materials. Thus, in various preferred aspects, the total endotoxin content of the BBI product is preferably no more than about 5.0 EU/g protein, no more than about 4.5 EU/g protein, no more than about 4.0 EU/g protein, no more than about 3.5 EU/g protein, no more than about 3.0 EU/g protein, no more than about 2.5 EU/g protein, no more than about 2.0 EU/g protein, no more than about 1.5 EU/g protein, no more than about 1.0 EU/g protein, and no more than about 0.5 EU/g protein. For example, in accordance with various such aspects, the total endotoxin content of the BBI product is typically from about 0.5 to about 5 EU/g protein, more typically from about 0.5 to about 2.5 EU/g protein and, still more typically, from about 0.5 to about 1 EU/g protein.

BBI proteins are known to inhibit both chymotrypsin and trypsin, while other components of the protein-containing composition (e.g. KTI proteins) are known to inhibit only trypsin. Thus, it is currently believed that the ratio of chymotrypsin inhibitor activity to trypsin inhibitor activity is an indicator of the presence of BBI proteins. Generally, the ratio of chymotrypsin inhibitor activity to trypsin inhibitor activity is at least about 1:1, at least about 1:2, at least about 1:3, at least about 1:4, at least about 1:5, at least about 1:6, at least about 1:7, at least about 1:8, at least about 1:9, or at least about 1:10. In specific aspects of the invention, the ratio of chymotrypsin inhibitor activity to trypsin inhibitor activity is about 1:1, about 1:1.1, about 1:1.2, about 1:1.3, about 1:1.4, about 1:1.5, about 1:1.6, about 1:1.7, about 1:1.8, or about 1:1.9.

Chymotrypsin inhibitor activity of BBI products of the present disclosure (expressed in terms of chymotrypsin inhibitor units/g protein, or CIU/g protein) may be determined by conventional methods known in the art. Generally, the chymotrypsin inhibitor activity of BBI products of the present disclosure is at least about 500 CIU/g protein, more generally at least about 1000 CIU/g protein, and still more generally at least about 1200 CIU/g protein. Typically, the chymotrypsin inhibitor activity of BBI products of the present disclosure is at least about 1600 CIU/g protein, at least about 2500 CIU/g protein, at least about 2700 CIU/g protein, or at least about 3000 CIU/g protein.

Chymotrypsin inhibitor activity is carried out as described previously (Ware et al., 1997 Arch. Biochem. Biophys. Vol 344, No. 1 pp. 133-138) with the following modifications. Alpha-Chymotrypsin from bovine pancreas was purchased from Sigma Chemical Co. (cat# C4129, St. Louis, Mo.) with the active chymotrypsin quantitated by active-site titration with methylumbelliferyl α-trimethylammoniocinnamate chloride (MUTMAC, cat# M5407, Sigma Chemical Co., St. Louis, Mo.) based on the method described by Jameson et al. (Biochem. J. 1973 131: 107-117). BBI samples were diluted to approximately 1 mg BBI/ml in deionized distilled (dl) H₂O (for example, weigh out purified BBI at 1 mg/ml, SWP at 10 mg/ml). In a siliconized microfuge tube the following were combined: a) BBI sample, 0-5ul; b) 0.1 M sodium phosphate and 1M NaCl at pH 7, 5ul; and c) 10 ul of 50 uM active chymotrypsin (dissolved in 1 mM HCl and 2 mM CaCl₂). Mix and incubate at room temperature for 10 minutes. To assay the residual chymotrypsin activity, dilute the sample 1:40 with dl H₂O, transfer 25 ul of diluted sample into a 1.5 ml glass cuvette containing 895 ul assay buffer (0.5M Tris, 20 mM CaCl₂, 1M NaCl, pH 8.0) and 80ul 10 mM sucAAPF-pNA (cat# S7388, Sigma chemical Co., St. Louis, Mo.), mix and immediately start measurement at Ab410 nm for 1 minute at 10 second intervals. Adjust the concentration of the inhibitor solution so that the results are obtained in the 40-80% inhibition range and extrapolate to determine the amount of sample needed to completely inhibit chymotrypsin. The chymotrypsin inhibition activity, (CI unit/g) is defined as the amount of sample which can completely inhibit 1 mg of active chymotrypsin as described previously in Ware et al. (1997 Arch. Biochem. Biophys. Vol 344, No. 1 pp. 133-138).

Similarly, trypsin inhibitor activity of BBI products of the present disclosure (expressed in terms of trypsin inhibitor units/g protein, or TIU/g protein) may be determined by conventional methods known in the art including, for example, in which one TIU is defined as the amount of a substrate which can inhibit 1 mg of trypsin and one trypsin unit equals ΔA₄₁₀ of 0.019 per 10 minute with benzoyl-DL-arginine-p-nitroanilide (BAPA) as substrate at pH 8.2 and 37° C. Generally, the trypsin inhibitor activity of BBI products of the present disclosure is at least about 400 TIU/g protein, more generally at least about 600 TIU/g protein, and still more generally at least about 800 TIU/g protein. Typically, the trypsin inhibitor activity of BBI products of the present disclosure is at least about 1000 TIU/g protein, at least about 1200 TIU/g protein, at least about 1400 TIU/g protein, or at least about 1600 TIU/g protein. Trypsin inhibitor activity is preferably no more than about 3000 TIU/g protein (i.e. theoretically pure).

It is to be understood that BBI products of the present disclosure may exhibit one, a combination, or all of the above-specified features. For example, BBI products of the present disclosure may exhibit the specified BBI purity and chymotrypsin inhibitor activity. BBI products may also exhibit the specified BBI purity, trypsin inhibitor activity or chymotrypsin inhibitor activity, and sequences disclosed herein. By way of further example, the BBI products may exhibit the specified BBI protein concentration and total endotoxin content. In these and still further aspects, the BBI products of the present disclosure may exhibit the specified total soy protein concentration and trypsin inhibitor activity. BBI products may also exhibit the specified total soy protein concentration and chymotrypsin inhibitor activity. By way of further example, BBI products of the present disclosure may exhibit the specified total soy protein concentration and total endotoxin content. These combinations of properties of the BBI products are exemplary and this list is not intended to be exhaustive. That is, in accordance with the present disclosure, BBI products may exhibit any combination of the above-noted properties, at any of the above-specified values of within any of the above-specified ranges.

BBI products of the present disclosure may be utilized in a variety of pharmaceutical compositions that may be included in a pharmaceutical preparation that is administered to a subject by at least one mode selected from the group consisting of oral, topical, parenteral, subcutaneous, intramuscular, intravenous, and intraperitoneal. In certain aspects of the invention, route of administration includes oral or parenteral. In other aspects of the invention, route of administration includes orally by way of a food. Depending on the desired duration and effectiveness of the therapy, the compositions according to the invention may be administered once or several times, also intermittently, for instance on a daily or weekly basis for several days, weeks, or months in different dosages and by a combination of different routes. The BBI products of the present disclosure may also be utilized in dietary supplement formulations. Suitable forms of pharmaceutical and dietary supplement compositions include, for example, syrups, powders, creams, injectibles, suspensions, emulsions, tablets, capsules, lozenges, suppositories, and mouthwashes.

In addition to various pharmaceutical applications, BBI products of the present disclosure are also suitable for incorporation into a wide variety of personal care products. For example, the BBI products of the present disclosure are currently believed to decrease photoaging of the skin (see, for example, Paine C. et al., J. Invest. Dermatol. 116: 587-595 (2001)) and therefore, are suitable for incorporation in cosmetic and skin care products.

A further aspect of the present invention is the provision of a food product comprising a BBI product described herein. Such food product may include, but is not limited to, a beverage, a food bar, or other consumable known to one of ordinary skill in the art such when the food product is consumed a BBI product described herein is also consumed.

In one embodiment, the food product may be a beverage. Preferred beverages include ready-to-drink (RTD) beverages or dry-blended beverages (DBB). The beverage may be a substantially cloudy beverage or a substantially clear beverage. Non-limiting examples of suitable beverages include milk-based beverages, milk analog beverages (e.g., soymilk, rice milk, etc), weight management beverages, protein shakes, meal replacement drinks, coffee-based beverages, nutritional drinks, energy drinks, infant formulas, fruit juice-based drinks, fruit drinks, fruit-flavored drinks, vegetable-based drinks, sports drinks, and the like. The pH of the beverage may range and may be acidic, neutral, or alkaline.

In another embodiment, the food product may be a food bar, such as a granola bar, a cereal bar, a nutrition bar, or an energy bar. In still another embodiment, the food product may be a cereal-based product. Non-limiting examples of cereal-based food products include breakfast cereals, pasta, breads, baked products (i.e., cakes, pies, rolls, cookies, crackers), and snack products (e.g., chips, pretzels, etc.). The edible material of a cereal-based food product may be derived from wheat (e.g., bleached flour, whole wheat flour, wheat germ, wheat bran, etc.), corn (e.g., corn flour, cornmeal, cornstarch, etc.), oats (e.g., puffed oats, oatmeal, oat flour, etc), rice (e.g., puffed rice, rice flour, rice starch), and so forth. In another embodiment, the food product may be a nutritional supplement. The nutritional supplement may be liquid or solid.

BBI of the present invention can be obtained from any source or any process which allows for the separation, isolation, or purification of BBI from a native plant-based matrix. By way of non-limiting example, a native plant-based matrix can be derived from leguminous or non-leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods (e.g., apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatropha, green algae, and mixtures thereof. In particular aspects of the invention, BBI is obtained from soy in various processing streams. Various soy processing streams include, for example, an aqueous soy extract stream (which is any stream in which the protein components of a soy stream are in the soluble form, such as from a defatted soy material), an aqueous soymilk extract stream (which is any stream from a whole or partially defatted soy material in which the protein components of a soy stream are in the soluble form), an aqueous soy whey stream (which is any whey stream resulting from the precipitation or salting out of storage proteins; the precipitation method can include heat as well as chemical processes), an aqueous soy molasses stream (which is any stream generated by the removal of water from an aqueous soy whey stream), an aqueous soy protein concentrate soy molasses stream (which is any stream from the alcohol extraction of soluble sugars from the soy protein concentrate process), an aqueous soy permeate stream (which is any stream resulting from the separation of different molecular weight protein fractions where the smaller molecular weight proteins pass through a membrane), and an aqueous tofu whey stream (which includes any whey stream resulting from a tofu coagulation process). The amount of BBI product isolated by the processes of the present invention may be as small as a gram (lab scale isolation) or may be several metric tons (industrial or large scale isolation).

### B. Process for Obtaining a Soy Whey Protein

It is understood by those skilled in the art of separation technology that there can be residual components in each stream since separation is never 100%. Further, one skilled in the art realizes that separation technology can vary depending on the starting raw material.

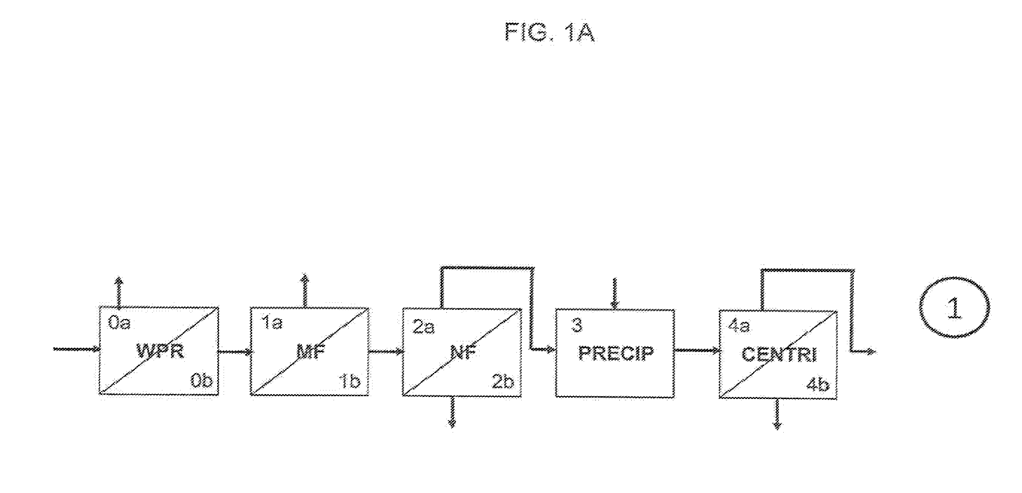

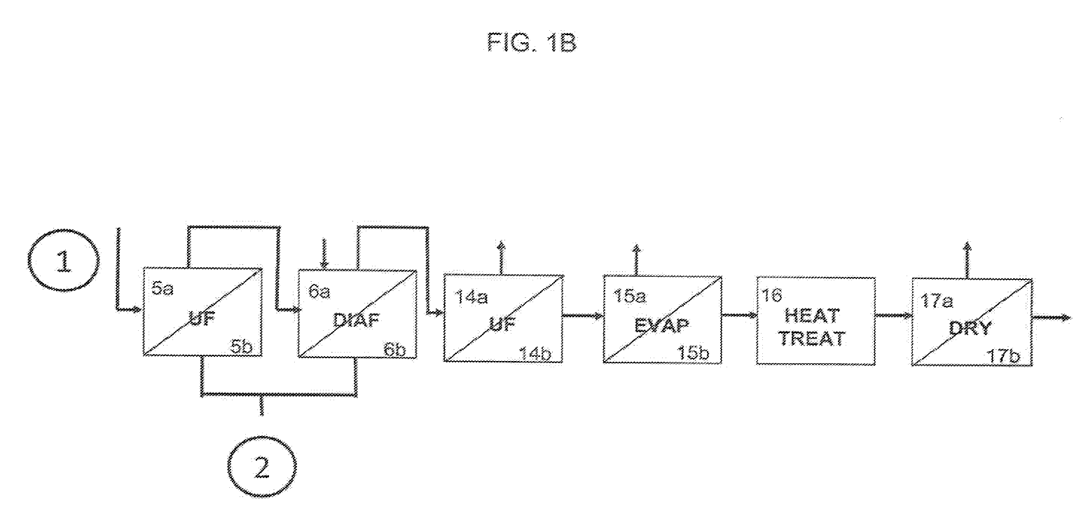

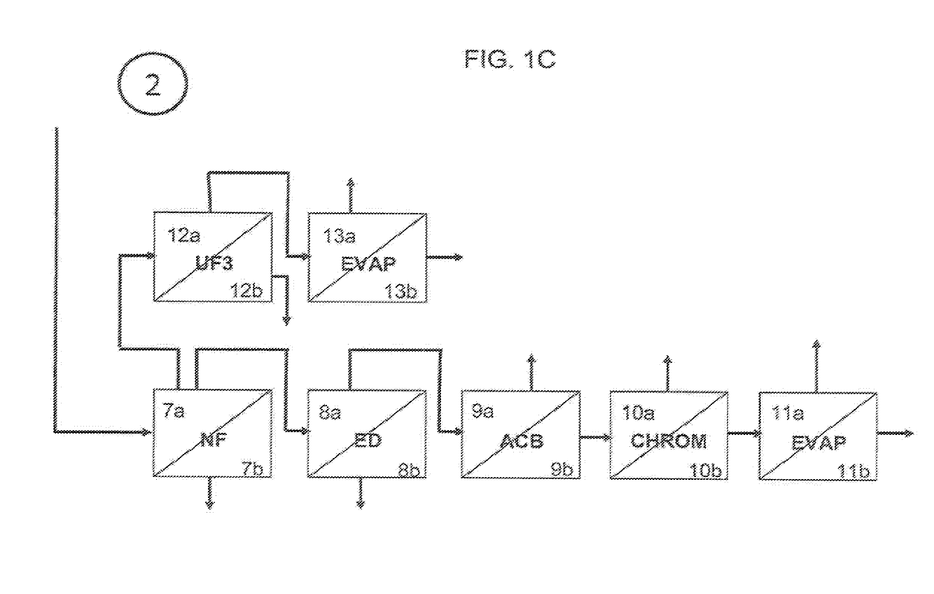

Step 0 (See FIG. 1A)—Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between 3.5 and 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kD)) in stream 0a and insoluble large molecular weight proteins (between about 300 kD and between about 50 kD) in stream 0b, such as pre-treated soy whey, storage proteins, and combinations thereof.

Step 1 (See FIG. 1A)—Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, or between about 3.5 and about 5.5, or about 5.3. The temperature can be between about 5° C. and about 90° C., or between about 25° C. and 75° C. or about 50° C. Products from step 1 include but are not limited to storage proteins, microorganisms, silicon, and combinations thereof in stream 1a and purified pre-treated soy whey in stream 1b.

Step 2 (See FIG. 1A)—A water and mineral removal can start with the purified pre-treated soy whey from stream 1b or 4a, or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and 75° C., or about 50° C. Products from this water removal step include but are not limited to purified pre-treated soy whey in stream 2a and water, some minerals, monovalent cations and combinations thereof in stream 2b.

Step 3 (See FIG. 1A)—the mineral precipitation step can start with purified pre-treated soy whey from stream 2a or pretreated soy whey from streams 0a or 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 1A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 1B)—the protein separation and concentration step can start with purified pre-treated whey from stream 4a or the whey from streams 0a, 1b, or 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 1B)—the protein washing and purification step can start with soy whey protein, BBI, KTI, storage proteins, other proteins or purified pre-treated whey from stream 4a or 5a, or whey from streams 0a, 1b, or 2a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., between about 25° C. and 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 7 (See FIG. 1C)—a water removal step can start with peptides, soy oligosaccharides, water, minerals, and combinations thereof from stream 5b and/or stream 6b. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., between about 25° C. and 75° C., or about 50° C. Products from stream 7a include but are not limited to, peptides, soy oligosaccharides, water, minerals, and combinations thereof. Products from stream 7b include but are not limited to, water, minerals, and combinations thereof.

Step 8 (See FIG. 1C)—a mineral removal step can start with peptides, soy oligosaccharides, water, minerals, and combinations thereof from streams 5b, 6b, 7a, and/or 12b. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., between about 25° C. and 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof.

Step 9 (See FIG. 1C)—a color removal step can start with de-mineralized soy oligosaccharides from streams 8a, 5b, 6b, 12b, and/or 7a). It utilizes an active carbon bed. Process variables and alternatives in this step include but are not limited to, ion exchange. Processing aids that can be used in this color removal step include but are not limited to, active carbon, ion exchange resins, and combinations thereof. The temperature can be between about 5° C. and about 90° C., or about 40° C. Products from stream 9a include but are not limited to, color compounds. Stream 9b is a decolored solution. Products from stream 9b include but are not limited to, soy oligosaccharides, and combinations thereof.

Step 10 (See FIG. C)—a soy oligosaccharide fractionation step can start with soy oligosaccharides, and combinations thereof from streams 9b, 5b, 6b, 7a, and/or 8a. It includes a chromatography step. Process variables and alternatives in this step include but are not limited to, chromatography, nanofiltration, and combinations thereof. Processing aids that can be used in this soy oligosaccharide fractionation step include but are not limited to acid or base to adjust the pH as one skilled in the art would know, based on the resin used. Products from stream 10a include but are not limited to, soy oligosaccharides. Products from stream 10b include but are not limited to soy oligosaccharides.

Step 11 (See FIG. 1C)—a water removal step can start with soy oligosaccharides from streams 9b, 5b, 6b, 7a, 8a, and/or 10b. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

Step 12 (See FIG. 1C)—an additional protein separation from soy oligosaccharides step can start with peptides, soy oligosaccharides, water, minerals, and combinations thereof from stream 7a, 5b, and/or 6b. It includes an ultrafiltration step. Process variables and alternatives in this step include but are not limited to, crossflow membrane filtration, ultrafiltration with pore sizes between about 50 kD and about 1 kD, and combinations thereof. Crossflow membrane filtration includes but is not limited to: spiral-wound, plate and frame, hollow fiber, ceramic, dynamic or rotating disk, nanofiber, and combinations thereof. Processing aids that can be used in this protein separation from sugars step include but are not limited to, acids, bases, protease, phytase, and combinations thereof. The pH of step 12 can be between about 2.0 and about 12.0, about 7.0. The temperature can be between about 5° C. and about 90° C., between about 25° C. and 75° C., or about 50° C. Products from stream 12b include but are not limited to, soy oligosaccharides, water, minerals, and combinations thereof. Products from stream 12a include but are not limited to, peptides, other proteins, and combinations thereof.

Step 13 (See FIG. 1C)—a water removal step can start with, peptides, and other proteins from stream 12a. It includes an evaporation step. Process variables and alternatives in this step include but are not limited to, reverse osmosis, nanofiltration, spray drying and combinations thereof. Products from stream 13a include but are not limited to, water. Products from stream 13b include but are not limited to, peptides, other proteins, and combinations thereof.

Step 14 (See FIG. 1B)—a protein fractionation step may be done by starting with soy whey protein, BBI, KTI, storage proteins, other proteins, and combinations thereof from streams 6a and/or 5a. It includes an ultrafiltration (with pore sizes from 300 kD to 10 kD) step. Process variables and alternatives in this step include but are not limited to, crossflow membrane filtration, ultrafiltration, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 14 can be between about 2.0 and about 12.0, or between about 6.0 and about 9.0, or about 7.0. The temperature can be between about 5° C. and about 90° C., between about 25° C. and 75° C., or about 50° C. Products from stream 14a include but are not limited to, storage proteins. Products from stream 14b include but are not limited to, soy whey protein, BBI, KTI, other proteins, and combinations thereof.

Step 15 (See FIG. 1B)—a water removal step can start with soy whey protein, BBI, KTI and, other proteins from streams 6a, 5a, and/or 14b. It includes an evaporation step. Process variables and alternatives in this step include but are not limited to, evaporation, nanofiltration, RO, and combinations thereof. Products from stream 15a include but are not limited to, water. Stream 15b products include but are not limited to soy whey protein, BBI, KTI, other proteins, and combinations thereof.

Step 16 (See FIG. 1B)—a heat treatment and flash cooling step can start with soy whey protein, BBI, KTI, other proteins from streams 6a, 5a, 14b, and/or 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Step 17 (See FIG. 1B)—a drying step can start with soy whey protein, BBI, KTI, other proteins from streams 6a, 5a, 14b, 15b, and/or 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI, other proteins, and combinations thereof.

### C. Aqueous Whey Streams

Aqueous whey streams and molasses streams, which are types of soy processing streams, are generated from the process of refining a whole legume or oilseed. The whole legume or oilseed may be derived from a variety of suitable plants. By way of non-limiting example, suitable plants include leguminous or non-leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods (e.g., apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatrophia, green algae, and mixtures thereof. In one embodiment, the leguminous plant is soybean and the aqueous whey stream generated from the process of refining the soybean is an aqueous soy whey stream.

Aqueous soy whey streams generated in the manufacture of soy protein isolates are generally relatively dilute and are typically discarded as waste. More particularly, the aqueous soy whey stream typically has a total solids content of less than about 10 wt. %, typically less than about 7.5 wt. % and, still more typically, less than about 5 wt. %. For example, in various aspects, the solids content of the aqueous soy whey stream is from about 0.5 to about 10 wt. %, from about 1 wt. % to about 4 wt. %, or from about 1 to about 3 wt. % (e.g. about 2 wt. %). Thus, during commercial soy protein isolate production, a significant volume of waste water that must be treated or disposed is generated.

Soy whey streams typically contain a significant portion of the initial soy protein content of the starting material soybeans. As used herein the term “soy protein” generally refers to any and all of the proteins native to soybeans. Naturally occurring soy proteins are generally globular proteins having a hydrophobic core surrounded by a hydrophilic shell. Numerous soy proteins have been identified including, for example, storage proteins such as glycinin and β-conglycinin. Soy proteins likewise include protease inhibitors, such as the above-noted BBI proteins. Soy proteins also include hemagglutinins such as lectin, lipoxygenases, β-amylase, and lunasin. It is to be noted that the soy plant may be transformed to produce other proteins not normally expressed by soy plants. It is to be understood that reference herein to “soy proteins” likewise contemplates proteins thus produced.

On a dry weight basis, soy proteins constitute at least about 10 wt. %, at least about 15 wt. %, or at least about 20 wt. % of the soy whey stream (dry weight basis). Typically, soy proteins constitute from about 10 to about 40 wt. %, or from about 20 to about 30 wt. % of the soy whey stream (dry weight basis). Soy protein isolates typically contain a significant portion of the storage proteins of the soybean. However, the soy whey stream remaining after isolate precipitation likewise contains one or more soy storage proteins.

In addition to the various soy proteins, the aqueous soy whey stream likewise comprises one or more carbohydrates (i.e. sugars). Generally, sugars constitute at least about 25%, at least about 35%, or at least about 45% by weight of the soy whey stream (dry weight basis). Typically, sugars constitute from about 25% to about 75%, more typically from about 35% to about 65% and, still more typically, from about 40% to about 60% by weight of the soy whey stream (dry weight basis).

The sugars of the soy whey stream generally include one or more monosaccharides, and/or one or more oligosaccharides or polysaccharides. For example, in various aspects, the soy whey stream comprises monosaccharides selected from the group consisting of glucose, fructose, and combinations thereof. Typically, monosaccharides constitute from about 0.5% to about 10 wt. % and, more typically from about 1% to about 5 wt. % of the soy whey stream (dry weight basis). Further in accordance with these and various other aspects, the soy whey stream comprises oligosaccharides selected from the group consisting of sucrose, raffinose, stachyose, and combinations thereof. Typically, oligosaccharides constitute from about 30% to about 60% and, more typically, from about 40% to about 50% by weight of the soy whey stream (dry weight basis).

The aqueous soy whey stream also typically comprises an ash fraction that includes a variety of components including, for example, various minerals, phytic acid, citric acid, and vitamins. Minerals typically present in the soy whey stream include sodium, potassium, calcium, phosphorus, magnesium, chloride, iron, manganese, zinc, copper, and combinations thereof. Vitamins present in the soy whey stream include, for example, thiamine and riboflavin. Regardless of its precise composition, the ash fraction typically constitutes from about 5% to about 30% and, more typically, from about 10% to about 25% by weight of the soy whey stream (dry weight basis).

The aqueous soy whey stream also typically comprises a fat fraction that generally constitutes from about 0.1% to about 5% by weight of the soy whey stream (dry weight basis). In certain aspects of the invention, the fat content is measured by acid hydrolysis and is about 3% by weight of the soy whey stream (dry weight basis).

In addition to the above components, the aqueous soy whey stream also typically comprises one or more microorganisms including, for example, various bacteria, molds, and yeasts. The proportions of these components typically vary from about 1×10² to about 1×10⁹ colony forming units (CFU) per milliliter. As detailed elsewhere herein, in various aspects, the aqueous soy whey stream is treated to remove these component(s) prior to protein recovery and/or isolation.

As noted, conventional production of soy protein isolates typically includes disposal of the aqueous soy whey stream remaining after isolation of the soy protein isolate. In accordance with the present disclosure, recovery of one or more proteins and various other components (e.g. sugars and minerals) results in a relatively pure aqueous whey stream. Conventional soy whey streams from which the protein and one or more components have not been removed generally require treatment prior to disposal and/or reuse. In accordance with various aspects of the present disclosure the aqueous whey stream may be disposed of or utilized as process water with minimal, if any, treatment. For example, the aqueous whey stream may be used in one or more filtration (e.g. diafiltration) operations of the present disclosure.

In addition to recovery of BBI proteins from aqueous soy whey streams generated in the manufacture of soy protein isolates, it is to be understood that the processes described herein are likewise suitable for recovery of one or more components of soy molasses streams generated in the manufacture of a soy protein concentrate, as soy molasses streams are an additional type of soy processing stream.

### D. Recovery of BBI Proteins

The processes described herein are directed to the recovery and isolation of purified BBI proteins present in an aqueous whey stream generated from the process of refining a whole legume or oilseed. As discussed hereinabove, the whole legume or oilseed may be derived from a variety of suitable plants. By way of non-limiting example, suitable plants include leguminous or non-leguminous plants, including for example, soybeans, corn, peas, canola, sunflowers, sorghum, rice, amaranth, potato, tapioca, arrowroot, canna, lupin, rape, wheat, oats, rye, barley, peanut, jack bean, Job's tears, pea family legumes, Baru, lablab beans, lancepods (e.g., apple leaf seed), alfalfa, snail medic seeds, lima beans, butter beans, kidney beans, bush beans, sugar cane, millet, timber tree, spinach, chapule, ciliates, dessert banana, lentil, bran, broad or fava bean, mung bean, adzuki bean, cow pea, jatrophia, green algae, and mixtures thereof. In one embodiment, the leguminous plant is soybean and the aqueous whey stream generated from the process of refining the soybean is an aqueous soy whey stream.

The present disclosure encompasses a variety of processes suitable for recovery of BBI proteins from aqueous soy whey streams generated in the production of soy protein isolates. Generally, the processes of the present disclosure comprise one or more operations designed and configured to separate out the particular components a soy processing stream (including, for example, an aqueous soy whey stream).

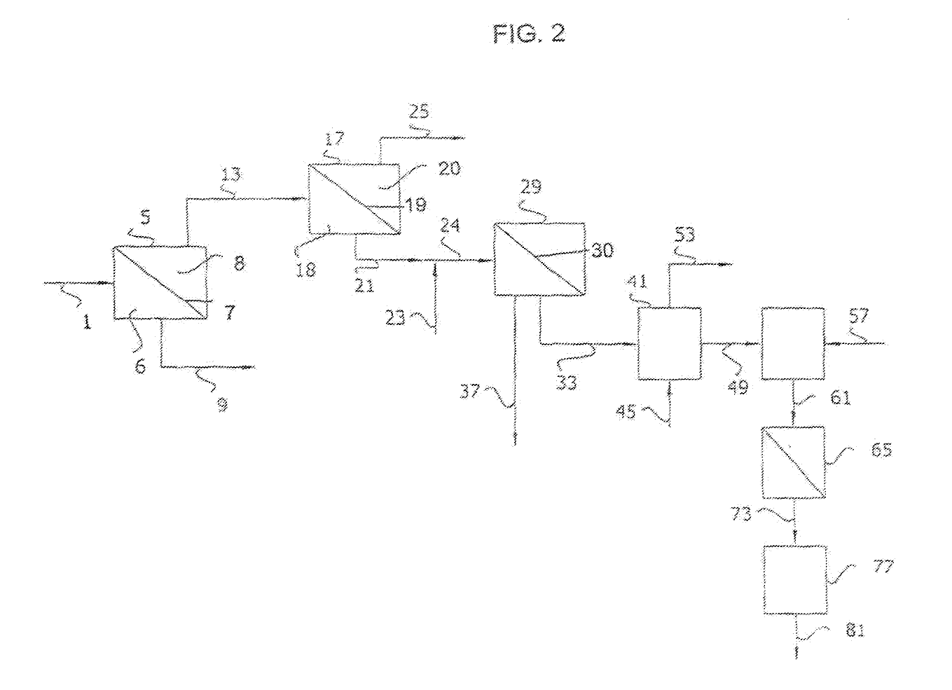

Generally, in accordance with the present disclosure, any of a variety of separation or purification techniques well-known in the art may be utilized to remove the various interfering components found in aqueous soy whey and isolate purified BBI proteins there from including, for example, membrane separation techniques (e.g. filtration, such as ultrafiltration, microfiltration, nanofiltration, and/or reverse osmosis), chromatographic separation techniques (e.g. ion exchange chromatography, adsorption chromatography, size exclusion chromatography, reverse phase chromatography, and affinity chromatography, which include, for example, anion or cation exchange chromatography, simulated moving bed chromatography, expanded bed adsorption chromatography, gel filtration, reverse-phase chromatography, ion exchange membrane chromatography, and mixed bed ion exchange chromatography), electrophoresis, dialysis, particulate filtration, precipitation, centrifugation, crystallization, and combinations thereof. A primary basis for separation of the various components is molecular size, although in filtration applications, the permeability of a filter medium can be affected by the chemical, molecular or electrostatic properties of the sample. As detailed elsewhere herein (e.g. below with reference to FIG. 2), processes of the present disclosure typically utilize more than one type of separation membrane depending upon the particular component of the whey stream to be removed. For example, one step of the process may utilize an ultrafiltration separation membrane, followed by one or more steps utilizing a nanofiltration separation membrane.

In various aspects, the present disclosure provides processes for recovery and isolation of purified BBI proteins present in an aqueous soy whey stream generated during soy protein isolate production. It should be noted that the processes of the present invention are not limited to soy whey or soy molasses streams and may be used to recover proteins and various other components from a wide variety of leguminous or non-leguminous plant processing streams. In various aspects, fractions comprising a high proportion of BBI proteins are recovered from the soy whey stream. For example, as detailed elsewhere herein, processes of the present disclosure provide BBI protein compositions having previously unachieved levels of purity.

Soy whey streams treated by the processes of the present disclosure are generally relatively dilute. To facilitate recovery and/or isolation of BBI proteins, the whey stream is preferably concentrated during the initial stage(s) of the process. Concentrating the soy whey stream aids in recovery and separation of BBI proteins from the whey stream. For example, in a preferred embodiment of the present disclosure, water is removed from the aqueous soy whey prior to recovery of BBI proteins by contacting the aqueous soy whey or a fraction thereof with a separation membrane to form a retentate comprising the aqueous soy whey and a permeate comprising water. In other embodiments of the present disclosure, water may be removed from the soy whey through any method known in the art, for example by evaporation.

Along with recovery of BBI proteins, processes of the present disclosure typically separate proteins from sugars present in the soy whey stream. Optionally, the processes of the present disclosure may be configured and controlled to separate the sugars of the soy whey stream into one or more fractions (e.g. a monosaccharide-rich fraction and/or an oligosaccharide-rich fraction). This may be done in multiple steps to separate different sugars from the proteins. Recovery of sugars from the soy whey stream thus provides a further product stream. As noted, sugar removal typically produces a fraction from which the sugars can be separated to yield both a concentrated sugar fraction and a relatively pure aqueous fraction that may be disposed of with minimal, if any, treatment or recycled as process water. Following treatment of the retentate to remove sugars, the retentate is further treated to remove additional components.

As noted, various soy whey streams that may be treated by the present disclosure include one or more minerals (e.g. phosphorus and calcium). It has been observed that the presence of one or more minerals may pose a challenge to downstream processing by, for example, membrane fouling and difficulty in separating from components desired to be recovered (i.e. BBI proteins). In addition to recovery of these desired components generally, removal of minerals from the soy whey is also currently believed to contribute to the recovery of BBI products having greater purity. As detailed elsewhere herein, mineral removal from the soy whey may generally proceed in accordance with methods known in the art including, for example, precipitation. Since phytic acid is typically present in the aqueous soy whey streams treated by the present processes, minerals such as calcium and magnesium are typically recovered in the form of calcium and magnesium phytates. Other minerals removed may also include, for example, sodium, potassium, zinc, iron, manganese, and copper.

In certain aspects for the removal of insoluble solids, particulate filtration, precipitation, centrifugation, crystallization, and combinations thereof may be used. Insoluble solids removed by these methods are typically greater than 5 microns.

Microfiltration is the process of separating solid particles from fluids by using a microfiltration membrane. Suitable microfiltration membranes are constructed of suitable materials known in the art including, for example, polysulfone, modified polysulfone, ceramic, and stainless steel. Microfiltration membranes typically have a pore size ranging from about 0.1 microns to about 5 microns. In certain aspects, microfiltration membranes have a pore size ranging from about 0.2 microns to about 2 microns.

Ultrafiltration is similar to microfiltration but differs in the pore size of the separation membrane. Ultrafiltration membranes are typically used to separate molecules having high molecular weights from molecules having lower molecular weights (including, for example, proteins). Suitable ultrafiltration membranes are typically constructed of suitable materials known in the art, such as, for example polysulfone (PS), polyethersulfone (PES), polypropylene (PP), polyvinylidenefluoride (PVDF), regenerated cellulose, ceramic, stainless steel, or thin-film composite. Ultrafiltration membranes typically have a molecular weight cut off (MWCO) of from about 1 to about 300 kilodaltons (kDa) or from about 5 to about 50 kDa. Additionally or alternatively, suitable ultrafiltration membranes may have a pore size of from about 0.002 microns to about 0.5 microns.

Nanofiltration is used to remove small molecules from fluids. Suitable nanofiltration membranes are typically constructed of suitable materials known in the art (e.g. polyethersulfone, polysulfone, ceramic, and polyamide-type thin film composite on polyester) and typically have a MWCO of from about 0.1 to about 5 kDa or from about 1 to about 4 kDa. Additionally or alternatively, suitable nanofiltration membranes may have a pore size of from about 0.9 nanometers to about 9 nanometers.

Reverse osmosis (or hyperfiltration) is typically used for the concentration of sugars. Suitable reverse osmosis membranes include those generally known in the art (e.g. membranes having a pore size of less 0.5 nm).

The separation membranes utilized in the filtration steps of the present invention may be arranged in accordance with one or more configurations known in the art, alone or in combination. For example, the membranes may be configured in the form a flat plate, or cassette module in which layers of membrane are combined together (along with optional layers of separator screens). Aqueous soy whey is generally introduced into alternating channels at one end of the stack and fluid passes through the membrane into one or more filtrate, or permeate channels. The separation membranes may also be arranged in a spiral wound module in which alternating layers of membrane are wound around a hollow central core. Aqueous soy whey is introduced into one end of the module while fluid passes through the alternating layers of the membrane and toward and into the core of the module. By way of further example, the separation membrane may be arranged in a hollow fiber module comprising a bundle of relatively narrow membrane tubes. Aqueous soy whey is introduced into the module and fluid passes through the bundle of membrane tubes transverse the flow of soy whey through the module. Suitable membrane arrangements are described, for example, in U.S. Pat. No. 6,946,075, the entire contents of which are incorporated herein by reference.

The filtration steps of the present invention, as further described herein, may utilize direct (normal-flow) filtration or tangential (cross-flow) filtration. In direct or normal-flow filtration, fluid (i.e. aqueous soy whey) is conveyed directly toward a separation membrane. Alternatively, in tangential or cross-flow filtration, fluid (i.e. aqueous soy whey) may be conveyed tangentially along the surface of the separation membrane. One advantage of tangential, or cross-flow filtration is that the frictional or sweeping force exerted tangentially on the membrane by the flow of aqueous soy whey typically aids in maintaining flux rate. Accordingly, in various aspects, one or more steps, and combinations thereof, in the processes of the present disclosure are operated as cross-flow filtration. Suitable cross-flow filters include those generally known in the art, including those described in U.S. Pat. No. 6,946,075. It is to be understood that passage of fluid may suitably proceed in accordance with normal and/or tangential (i.e. cross) flow. It is to be further understood that passage of fluid through other membrane separation units detailed elsewhere herein in connection with the embodiment depicted in FIG. 2, and other aspects, may proceed in accordance with either or both of these mechanisms.

The process described by the present invention involves selection of the appropriate separation operation or combination of operations to sequentially remove various constituents from the soy whey stream and recover or isolate a purified BBI product, which BBI product comprises a level of purity that has not been previously achieved in the art. In certain aspects of the invention, and as detailed elsewhere herein, the processes for recovery of BBI proteins utilize a combination of membrane separation and chromatographic separation (e.g. ion exchange) operations. In various aspects, recovery of individual BBI proteins proceeds by a simulated moving bed operation (often referred to in the art as an “SMB” configuration).

As noted elsewhere herein, aqueous soy whey streams treated by the processes of the present disclosure are generally relatively dilute. In various aspects the aqueous soy whey is concentrated by, for example, removal of water by a factor of at least about 2 (e.g. about 3 or about 6) prior to recovery of targeted, individual proteins.

As compared to other methods for recovery of BBI proteins, using simulated moving bed for recovery of non-BBI proteins generally may also provide advantages of lower cost, throughput, and/or flexibility due, at least in part, to the adaptability for treatment of plurality of samples of aqueous soy whey.

It has been observed that one or more components of the soy whey stream may interfere with recovery of BBI proteins. For example, often during soy protein isolate manufacture, a silicon compound, typically a silicone, is introduced as a defoaming agent, usually in the form of a silicon-containing compound such as those commercially available from Hydrite Chemical or Emerald Performance Materials. Regardless of the precise source, organic silicon compounds are typically present in the soy whey stream at concentrations of up to about 15 parts per million (ppm), up to about 10 ppm, or up to about 5 ppm based on silicon content. The presence of organic silicon compounds is generally undesired as it may interfere with recovery of BBI proteins of the soy whey stream.

Accordingly, in various aspects, silicones and/or other organic silicon compounds are removed from the soy whey stream as detailed elsewhere herein prior to treatment for recover and separation of BBI proteins. Preferably, silicon compounds are removed as further detailed herein to such a degree that the soy whey contains no more than trace levels of organic silicon. Additionally or alternatively, the aqueous soy whey may comprise one or more microorganisms that may interfere with recovery of the desired components of the aqueous soy whey and/or are undesired in a final, recovered product of the process.

For removal of these interfering components, the soy whey stream may be filtered using a separation membrane selective for retention of silicon defoaming agent and/or one or more microorganisms, to yield a retentate comprising silicon and/or one or more microorganisms and a permeate comprising the aqueous soy whey. The particular membrane (including, for example, microfiltration) used in this initial purification is selected in view of the component(s) to be removed. Regardless of the type of membrane selected and the component removed from the soy whey stream, preferably at least a substantial portion, and preferably substantially all, of the desired BBI protein is found in the retentate. Further in this regard, it is to be noted that reference to a permeate comprising the aqueous soy whey indicates that treatment of the whey stream for removal of one or more impurities has little, if any, impact on the other components of the soy whey stream.

In various alternative aspects, bacteria contained in the whey stream may be killed by heating prior to recovery of proteins. The manner of heating the soy whey stream for destroying bacteria is not narrowly critical and may generally be conducted in accordance with conventional methods known in the art. However, heating the soy whey stream for destruction of microorganisms may introduce a risk of protein denaturation. Accordingly, removal of bacteria and other microorganisms from the soy whey stream by methods that do not include heating the soy whey stream are generally preferred.

FIG. 2 depicts an embodiment of a process of the present disclosure for recovery of one or more individual proteins from a soy whey stream generated in the production of soy protein isolate.

As illustrated in FIG. 2, an aqueous soy whey 1 is introduced into a membrane separation unit 5 comprising a first filtration feed zone 6 in contact with one side of a separation membrane 7 at a pressure higher than the pressure in a first permeate zone 8 on the other side of the membrane. Preferably, membrane separation unit 5 comprises at least one microfiltration membrane.

The transmembrane pressure across the separation membrane 7 within membrane separation unit 5 is generally at least about 5 psi, at least about 25 psi, at least about 50 psi, at least about 100 psi, or at least about 150 psi. Fluid typically passes through the membrane at a volumetric flow, or flux of at least about 1 liter fluid/hour-m², or from about 1 to about 200 liters fluid/hour-m² cross-sectional membrane area transverse to the direction of flow. Flow rate may be affected by, for example, the type of filtration, fouling of membranes, etc. The soy whey is typically introduced into the filtration feed zone of the membrane separation unit at a temperature of from about 0° C. to about 100° C. and, more typically, at a temperature of from about 25° C. to about 60° C. Typically, aqueous soy whey 1 is reduced in volume by about 5% due to the retentate.

Passage of fluid through the separation membrane results in a first retentate 9 and a first permeate 13 within first permeate zone 8. The first retentate 9 will primarily comprise one or more microorganisms and insoluble material, more particularly, the first retentate 9 typically is enriched in microorganisms relative to the first permeate 13. Preferably, the first retentate 9 contains a substantial portion, if not substantially all, of the microorganism content of the aqueous soy whey. Even more preferably, the first retentate 9 also comprises a substantial portion of the antifoam agent (e.g. silicon of the organic silicon- or lipid-containing containing compounds present in the aqueous soy whey) and, more particularly, preferably comprises at least about 70 wt. %, more preferably at least about 80 wt. % and, still more preferably, at least about 90 wt. % of the antifoam agent content of the aqueous soy whey based on antifoam agent content. The first permeate 13 will primarily comprise all of the various remaining components of the aqueous soy whey stream, such as the soluble soy storage proteins, soy whey proteins, various sugars, water, minerals, isoflavones, and vitamins.

Again with reference to FIG. 2, the first permeate 13 is introduced into membrane separation unit 17 comprising a second filtration feed zone 18 in contact with one side of a separation membrane 19 at a pressure higher than the pressure in a second permeate zone 20. Membrane separation unit 17 preferably comprises at least one ultrafiltration membrane as the separation membrane 19. The transmembrane pressure across the separation membrane 19 within membrane separation unit 17 is generally at least about 5 psi, at least about 10 psi, at least about 25 psi, at least about 50 psi, at least about 100 psi, or at least about 150 psi. Fluid typically passes through the membrane at a volumetric flow, or flux, of at least about 1 liter fluid/hour-m², or from about 1 to about 150 liters fluid/hour-m² cross-sectional membrane area transverse to the direction of flow. The soy whey is typically introduced into the filtration feed zone of the membrane separation unit at a temperature of from about 0° C. to about 100° C. and, more typically, at a temperature of from about 25° C. to about 60° C. Typically, aqueous soy whey 1 is concentrated by a concentration factor of at least about 5, or from about 5 to about 75 (e.g. about 25). The ultrafiltration step may optionally include diafiltration. Diafiltration volumes may typically range from about 1 up to about 10 parts diafiltration volume per part of retentate.

Passage of fluid through the separation membrane results in a second retentate 21 and a second permeate 25. The second retentate 21 comprises a significant fraction of the protein content of the aqueous soy whey and, thus, is further treated for recovery of BBI proteins. Preferably, the second retentate 21 comprises at least about 25 wt. % to at least about 90 wt. % (e.g. at least about 50 wt. %) (dry weight basis) of various soy whey proteins present in the aqueous soy whey introduced into the first filtration feed zone 6.

Again with reference to FIG. 2, the second permeate 25 generally comprises any proteins not recovered in second retentate 21 and various other components of the soy whey stream (e.g. various sugars, water, minerals, vitamins, and isoflavones). Although not illustrated in FIG. 2, the components of the second permeate 25 may be further processed according to suitable separation operations in order to isolate and/or remove the individual components from the aqueous whey stream. Following the additional separation steps, a relatively pure water stream will preferably be formed, requiring minimal, if any, treatment prior to disposal or use. Therefore, the invention described herein also possesses environmental benefits by, for example, improving environmental quality.

The second retentate 21 is combined with a carrier stream 23 to form the feed 24 to the ion exchange column or unit 29 containing at least one ion exchange resin 30. The precise composition of the carrier stream is not narrowly critical. The pH of the carrier stream is from about 1 to about 7, more typically from about 2 to about 6 and, still more typically, from about 2 to about 5. In various aspects for recovery of BBI proteins, the carrier stream comprises a non-volatile buffer, including, for example, sodium citrate, or a volatile buffer including, for example, ammonium formate. For example, in various aspects, the carrier stream comprises a counter ion containing buffer in an aqueous mixture at a concentration of from about 10 to about 30 millimolar (e.g. 20 mM).

The pH of the second retentate 21 and/or feed stream 24 affects solubility of soy proteins, and precipitated proteins may result in fouling of the ion exchange resin. Thus, it may be desired to control the pH of the feed to the ion exchange column within certain limits (e.g. by buffering). If necessary, the pH of the feed may be maintained within the ranges by, for example, dilution of the second retentate, carrier stream, and/or the feed provided by the combination of the retentate and carrier stream. The composition of the diluent is not narrowly critical and is typically an aqueous medium (e.g. deionized water) that may be readily selected by one skilled in the art. In addition to impacting the pH of the feed, dilution also typically reduces the inherent ionic strength of the feed, which promotes binding of proteins to the ion exchange resin. Additionally or alternatively, the pH of the feed may be controlled by selection of the carrier stream.

The ion exchange resin is chosen to be suitable for selective retention and recovery of one or more proteins present in second retentate 21 and feed 24. In various aspects, the ion exchange resin is selected for selective retention of BBI proteins or retention of non-BBI proteins such that BBI proteins are separated from non-BBI proteins. The following discussion focuses on recovery and isolation of BBI proteins from an aqueous soy whey (i.e. second retentate 21). However, it is to be understood that the following procedure is readily adaptable to recovery of other target proteins (e.g. KTI proteins) as well as other types of incoming streams besides aqueous (e.g. reconstituted from spray dried).

Regardless of the precise configuration of the ion exchange unit, suitable ion exchange resins for recovery of BBI proteins include a variety of cation and anion exchange resins. Although both cation exchange resins and anion exchange resins are, depending on the feed to the ion exchange column, suitable for recovery of BBI proteins, in various aspects the ion exchange resin comprises a cation exchange resin. For example, a protein exposed to a pH below its isoelectric point (pI) is more likely to have regions of positive charge and, therefore, bind more tightly to a cation exchange resin. Most proteins in the feed stream have a pI higher than that of BBI and the typical pH of the feed. Therefore, these proteins typically bind more tightly to the resin. A BBI protein-containing fraction may be readily eluted from the ion exchange column by contacting the resin with a suitable eluant.

Alternatively, the pH of the feed may be controlled to be below the pI of BBI protein to provide retention of BBI proteins by the ion exchange resin. Other proteins (e.g. KTI proteins) are also bound to the resin. However, recovery of desired fractions may proceed by contacting the ion exchange resin with a suitable eluant for differential elution of protein fractions.

Suitable cation exchange resins include a variety of resins well-known in the art. In at least one embodiment, the ion exchange resin comprises a Poros 20 HS—a cross-linked poly(styrene-divinylbenzene) matrix which is surface coated with a polyhydroxylated polymer functionalized with sulfopropyl groups (e.g. propylsulfonic acid, —CH₂CH₂CH₂SO₃⁻) manufactured by Applied Biosystems.

It has been observed that adjusting the pH of the retentate and/or feed may result in precipitation of non-BBI proteins. In such instances, the precipitated proteins may be separated from the feed (not shown in FIG. 2) by any membrane separation technique (e.g. filtration, such as ultrafiltration, microfiltration, nanofiltration, and/or reverse osmosis), chromatographic separation technique (e.g., ion exchange chromatography, adsorption chromatography, size exclusion chromatography, reverse phase chromatography, and affinity chromatography, which include, for example, anion or cation exchange chromatography, simulated moving bed chromatography, expanded bed adsorption chromatography, gel filtration, reverse-phase chromatography, ion exchange membrane chromatography, and mixed bed ion exchange chromatography), electrophoresis, dialysis, particulate filtration, precipitation, centrifugation, crystallization, gravity separation (including salting out or salting in using, for example, ammonium sulfate or ammonium chloride, respectively) or combination thereof prior to introduction into the ion exchange column. Separation may be carried out from about 0° C. to 100° C. at a pH range from about 1 to 10.

Again with reference to FIG. 2, after passage of feed 24 through the ion exchange column 30, an eluted BBI protein stream 33 is recovered.

If necessary, the ion exchange resin is contacted with a suitable eluant(s) to yield an eluted BBI protein-containing stream 33 and a KTI protein-containing stream 37. Elution of proteins from the ion exchange column typically proceeds via a multi-stage process. In accordance with various aspects, in a first stage the column is contacted with an eluant for removal of BBI proteins from the ion exchange resin. Suitable eluants include, for example, mixtures of sodium chloride and sodium citrate. For example, suitable eluants can include mixtures of sodium chloride and sodium citrate at a volumetric ratio of sodium chloride to sodium citrate of from about 15:1 to about 25:1 using between 1 mM and 400 mM solutions. In addition to BBI proteins thus eluted, the BBI protein-containing stream can pass through the ion exchange column (i.e., flow through) depending on the conditions (e.g., pH, ionic strength, etc.). Elution buffers include, for example, a buffer and appropriate counter-ion, which can be determined by one of ordinary skill in the art. In a second stage, the ion exchange resin is contacted with an eluant for removal of non-BBI proteins in the form of, for example, a KTI protein-containing stream 37.

In certain aspects of the invention whereby BBI proteins are obtained using an ion exchange column that does not retain BBI proteins (i.e., flow through), BBI proteins bear the same charge as the stationary phase of the column and as a result flow through without being retained. However, non-BBI proteins are retained by the column.

Again with reference to FIG. 2, the BBI protein-containing stream 33, along with a liquid precipitating medium 45, is introduced into a separation unit 41 comprising a precipitation zone. Generally, the liquid precipitating medium 45 comprises a precipitating agent. Typically, the liquid precipitating agent comprises ammonium sulfate to precipitate BBI protein from the BBI protein stream 33. In various aspects, the liquid precipitating medium comprises ammonium sulfate at a concentration of from about 30% to about 60% (e.g. from about 40% to about 50%) of its saturation concentration in the liquid precipitating medium.

Contact of the BBI protein fraction 33 with the precipitating medium 45 within the precipitation zone forms a precipitated BBI protein fraction 49 and supernatant 53 that are removed from the separation unit 41. The precipitated BBI protein fraction 49 is combined with an aqueous washing medium 57 in the presence or absence of salts or buffers to form a solubilized BBI protein fraction 61. This BBI protein fraction 61 may comprise residual precipitating agent (e.g. ammonium sulfate) and one or more other impurities.

As illustrated in FIG. 2, the solubilized protein fraction 61 is introduced into a dialysis or diafiltration unit 65 for removal of any residual precipitating agent and one or more impurities. The form and configuration of the dialysis or diafiltration unit are not narrowly critical and the unit may be readily selected by one skilled in the art. For example, a dialysis unit comprising suitable dialysis cassettes (e.g. Slide-A-Lyzer, manufactured by Thermo Scientific Pierce Protein Research Products having a molecular weight cutoff of 2000 Daltons) or a diafiltration unit comprising a cross flow filtration membrane (which may be more suitable for large scale separation) may be utilized. Removal of residual precipitating agent and/or impurities is determined by monitoring the conductivity of the solubilized protein fraction 61. Once suitable impurity removal is achieved, a purified BBI solubilized protein fraction 73 is removed from the dialysis or diafiltration unit 65. The purified solubilized BBI protein fraction 73 may be introduced into a drying unit 77 (e.g. lyophilization unit or a spray dryer unit) to form a dry, purified BBI protein product 81. Optionally, treatment of the purified solubilized BBI protein fraction 73 removes one or more remaining impurities from the BBI protein fraction to form the purified BBI protein product 81 of the present invention. For example, treatment with Triton® X114 is used for removal of one or more endotoxins.

FIG. 3 illustrates the SDS-PAGE purity analysis of the various retentates and permeates isolated during the process of the invention as depicted in FIG. 2, including the final BBI product. Lane 1 depicts the composition of the soy whey prior to separation and indicates the presence of multiple components. In contrast, lane 8 depicts the BBI protein isolated from the soy whey following the separation process of the present invention and is virtually free of additional components, which indicates a high level of purity.

The process scheme depicted in FIG. 2 is not limited to the starting material used or to the order of separation and recovery of components of the soy whey set forth above, and may be utilized to prepare process streams differing from those discussed above including, for example, as set forth in the appended claims.

### E. Additional Methods of Making a BBI Protein

In certain embodiments, a BBI protein of the invention is produced by, for example, recombinant means or synthetically. Recombinant production of a protein of the invention is done using standard techniques known by one of ordinary skill in the art. Such methods include, for example, producing a one or more coding nucleic acid sequences, which can be done by polymerase chain reaction (PCR) based methods using as a template the full-length cDNA sequence. Following production of the desired nucleic acid sequence, the sequence is inserted into an expression plasmid (including, for example, Escherichia coli pCAL-n expression plasmid), which is then transfected in a microorganism; then selection of clones containing a plasmid containing the desired sequence using selection markers (including, for example, an antibiotic resistance selection marker or a luminescent selection marker) is performed; followed by mass producing clones containing a plasmid containing the desired sequence; and purifying peptides from the desired clones (see, for example, methods described Gorlatov et al. Biochemistry (2002) 41, 4107-4116; U.S. Pat. No. 4,980,456). Alternatively peptides of the invention can be made by synthetic means or semi-synthetic means (e.g., a combination of recombinant production and synthetic means).

Synthetic production can be done by, for example, applying a fluorenylmethyloxycarbonyl(FMOC)-protective group strategy according to Carpino L. A. and Han. G Y, J. (Amer. Chem. Soc. 1981; 37; 3404-3409) or a tert-butoxycarbonyl(t-Boc)-protective group strategy. Peptides are synthesized, for example, by means of a solid-phase peptide synthesis according to Merrifield R. B. (J. Amer. Chem. Soc. 1963; 85, 2149-2154), using a multiple peptide synthesizer. Crude peptides are then purified.

An exemplary method for the synthetic production of a protein of the invention is described in the following passage. 100 mg Tentagel-S-RAM (Rapp-Polymere) at a load of 0.24 mmol/g is transferred to a commercially available peptide synthesis device (PSMM(Shimadzu)), wherein the peptide sequence is constructed step-by-step according to the carbodiimide/HOBt method. The FMOC-amino acid derivatives are pre-activated by adding a 5-fold equimolar excess of di-isopropy-carbodiimide (DIC), di-isopropy-ethylamine (DIPEA) and hydroxybenzotriazole (HOBt), and following their transfer into the reaction vessel, mixed with the resin support for 30 minutes. Washing steps are carried out by, for example, additions of DMF and thorough mixing for 1 minute. Cleavage steps are carried out by, for example, the addition of piperidine in DMF and thorough mixing for 4 minutes. Removal of the individual reaction and wash solutions is effected by forcing the solutions through the bottom frit of the reaction vessel. The amino acid derivatives FMOC-Ala, FMOC-Arg(Pbf), FMOC-Asp, FMOC-Gly, FMOC-His(Trt), FMOC-IIe, FMOC-Leu, FMOC-Lys(BOC), FMOC-Pro, FMOC-Ser(tBu) and FMOC-Tyr(tBu) (Orpegen) are employed. When synthesis is completed the peptide resin is dried. The peptide amide is subsequently cleaved off by treatment with trifluoracetic acid/TIS/EDT/water (95:2:2:1 vol) for 2 hours at room temperature. By way of filtration, concentration of the solution and precipitation by the addition of ice-cold diethyl ether, the crude product is obtained as a solid. The peptide is then purified by RP-HPLC in 0.1% TFA with a gradient of 5 on 60% acetonitrile in 40 minutes at a flow rate of 12 ml/min and evaluation of the elutant by means of a UV detector at 215 nm. The purity of the individual fractions is determined by analytical RP-HPLC and mass spectrometry.

### F. Methods of Use

In certain aspects of the invention, a BBI protein of the invention is used to treat certain pathological states in a subject. A BBI protein is a single BBI protein or any mixture of BBI proteins described herein. In certain aspects a mixture of BBI proteins comprises two or more of any of SEQ ID NO: 1, SEQ ID NO: 2, SEQ ID NO: 3, SEQ ID NO: 4, SEQ ID NO: 5, or SEQ ID NO: 6. In other aspects a mixture of BBI proteins comprises three or more of any of SEQ ID NO: 1, SEQ ID NO: 2, SEQ ID NO: 3, SEQ ID NO: 4, SEQ ID NO: 5, or SEQ ID NO: 6. In further other aspects a mixture of BBI proteins comprises four or more of any of SEQ ID NO: 1, SEQ ID NO: 2, SEQ ID NO: 3, SEQ ID NO: 4, SEQ ID NO: 5, or SEQ ID NO: 6. In even further other aspects a mixture of BBI proteins comprises five or more of any of SEQ ID NO: 1, SEQ ID NO: 2, SEQ ID NO: 3, SEQ ID NO: 4, SEQ ID NO: 5, or SEQ ID NO: 6. In yet even further other aspects a mixture of BBI proteins comprises SEQ ID NO: 1, SEQ ID NO: 2, SEQ ID NO: 3, SEQ ID NO: 4, SEQ ID NO: 5, and SEQ ID NO: 6. Generally, when administering proteins for a therapeutic purpose the proteins are administered in a composition. In this aspect of the invention and other aspects of the invention drawn to a composition, a composition of the invention comprises, consists essentially of, or consists of a BBI protein.

The pathological states that can be treated by a BBI protein of the invention range from diseases associated with muscle dysfunction to uncontrolled cell growth. Provided herein are some specific examples of pathological states that can be treated by the invention. The examples discussed are for illustration purposes only. The uses contemplated are not restricted to the specific examples described herein.

In certain aspects of the invention, a BBI protein of the invention can be used to treat skeletal muscle atrophy (including, for example, sarcopenia), treat skeletal muscle wasting (including, for example, cachexia), treat skeletal muscle degeneration, improve skeletal muscle function, or treat a degenerative skeletal muscle disorder or disease, including, for example, muscular dystrophy, amyotrophic lateral sclerosis, spinal muscle atrophy and spinal cord injury. Other muscle diseases and experimental methods associated therewith can be found in U.S. Patent Application Publication No. 20080300179 (see also Morris et al., J Appl Physiol. 2005 November; 99(5):1719-27. Epub 2005 Jun. 23; Arbogast et al., J Appl Physiol. 2007 March; 102(3):956-64. Epub 2006 Nov. 16).

In other aspects of the invention, a BBI protein of the invention can be used to treat an autoimmune disease, including, for example, rheumatoid arthritis, diseases characterized by neuroinflammation and/or demyelination (including, for example, multiple sclerosis and Guillain Barre syndrome), autoimmune immunodeficiency, chronic fatigue syndrome, systemic and discoid lupus, celiac disease, Crohn's disease, inflammatory bowel disease, and ulcerative colitis. Other autoimmune diseases and experimental methods associated therewith can be found in U.S. Patent Application Publication Nos. 20040142050 and 20020127289 (see also Touil et al., J Neurol Sci. 2008 Aug. 15; 271(1-2):191-202. Epub 2008 Jun. 10; Lichtenstein et al., Dig Dis Sci. 2008 January; 53(1):1 75-80. Epub 2007 Jun. 6).

In other aspects of the invention, a BBI protein of the invention can be used to treat a cell proliferative disorder, including for example, a cancer. A cancer includes for example, colon cancer, prostate cancer, blood-related cancer (including, for example, leukemia), bone cancer, urogenital cancer, peripheral and central nervous system cancer, gastrointestinal cancer, breast cancer, head and neck cancer, oral cancer, liver cancer, lung cancer, pancreatic cancer, oral leukoplakia, and viral induced cancers (including, for example, HPV-induced cancer). Other cancers and experimental methods associated therewith can be found in U.S. Patent Application Publication Nos. 20080248566; 20020198247; 20020187978; 20020183388; 20020173468; 20020156292; and U.S. Pat. Nos. 5,338,547; 5,505,946; 5,962,414; 5,961,980; 6,323,205; 6,319,923; and 6,284,239 (see also Saito et al., Cancer Lett. 2007 Aug. 18; 253(2):249-57. Epub 2007 Mar. 6; Dittmann et al., Radiother Oncol. 2008. March; 86(3):375-82. Epub 2008 Jan. 30).

In other aspects of the invention, a BBI protein of the present invention can be used to treat a skin disorder or promote skin health. Examples of skin disorders include skin discoloration or pigmentation and inflammatory skin conditions, including for example, atopic dermatitis. Examples of promoting skin health include reduction in skin discoloration or pigmentation, reduction in inflammation, wrinkle minimization, wrinkle removal, decoloring, coloring, skin softening, skin smoothing, depilation, and cleansing. Other examples of skin health and experimental methods associated therewith can be found in U.S. Patent Application Publication Nos. 20100093028, 20100092409, 20090111160 (see also Paine et al. J. Invest Dermatol. 2001 April; 116(4):587-95).

### DEFINITIONS

To facilitate understanding of the invention, several terms are defined below.

The term “acid soluble” as used herein refers to a substance having a solubility of at least about 80% with a concentration of 10 grams per liter (g/L) in an aqueous medium having a pH of from about 2 to about 7.

The terms “soy protein isolate” or “isolated soy protein,” as used herein, refer to a soy material having a protein content of at least about 90% soy protein on a moisture free basis.

The term “subject” or “subjects” as used herein refers to a mammal (preferably a human), bird, fish, reptile, or amphibian, in need of treatment for a pathological state, which pathological state includes, but is not limited to, diseases associated with muscle, uncontrolled cell growth, autoimmune diseases, and cancer.

The term “processing stream” as used herein refers to the secondary or incidental product derived from the process of refining a whole legume or oilseed, including an aqueous stream, a solvent stream, or a reconstituted from dried (e.g., spray dried) stream, which includes, for example, an aqueous soy extract stream, an aqueous soymilk extract stream, an aqueous soy whey stream, an aqueous soy molasses stream, an aqueous soy protein concentrate soy molasses stream, an aqueous soy permeate stream, an aqueous tofu whey stream, and additionally includes soy whey protein, for example, in both liquid and dry powder form, that can be recovered as an intermediate product in accordance with the methods disclosed herein.

The term “other proteins” as used herein is defined as including, but not limited to, lunasin, lectins, dehydrins, lipoxygenase, and combinations thereof.

The term “soy whey protein” or “soy whey” as used herein is defined as including proteins soluble at those pHs where soy storage proteins are typically insoluble including, but not limited to, BBI, KTI, lunasin, lipoxygenase, dehydrins, lectins, peptides, and combinations thereof. Soy whey protein may further include storage proteins.

The term “soy oligosaccharides” as used herein is defined as including, but not limited to, sugar. Sugar is defined as including but not limited to sucrose, raffinose, stachyose, verbascose, monosaccharides, and combinations thereof.

When introducing elements of the present invention or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

As various changes could be made in the above compounds, products and methods without departing from the scope of the invention, it is intended that all matter contained in the above description and in the examples given below, shall be interpreted as illustrative and not in a limiting sense.

### EXAMPLES

### Example 1

### Recovery of BBI protein from soy whey protein

Aqueous soy whey (145 I) having a total solids content of approximately 3.7 wt. % and a total protein content of 22.5 wt. % (dry wt. basis) was introduced into an OPTISEP 7000 filtration module containing a BTS-25 or MMM 0.45 micron microfiltration membrane. Passage of the aqueous soy whey through the membrane formed a permeate (132 I having a solids content of 3.2 wt. %) containing aqueous soy whey and a retentate containing greater than 99% of the initial bacteria content of the soy whey and greater than 90% of the silicon defoamer content of the soy whey.

Permeate (132 I) from the microfiltration module was introduced into an OPTISEP 7000 filtration module containing a regenerated cellulose (RC) ultrafiltration membrane having a pore size of approximately 100 kDa. Passage of the permeate through the ultrafiltration membrane formed a second permeate containing sugars, minerals, and vitamins, and a second retentate (approx. 2 I) having a solids content of approximately 25.4 wt. % and a total soy protein content of approximately 83 wt. % (dry basis).

Second retentate (516 ml) was introduced in small batches into an ion exchange column containing a Poros 20 HS cation exchange resin (68.3 ml bed volume), which was pre-equilibrated with 20 mM sodium citrate at pH 3. The pH of the retentate contacted with the ion exchange resin (i.e. feed stream introduced into the ion exchange column) was maintained at about 4.15 by 5× dilution with 20 mM sodium citrate pH 3 and addition of HCl, as necessary.

Passage of each batch of the retentate through the column at a linear flow rate of approximately 76 cm/hr yielded a BBI protein stream (approx. 73g). A second protein fraction (approx. 9g) containing BBI proteins was recovered from the ion exchange column by elution with 400 mM sodium chloride in 20 mM sodium citrate pH 3 and a third protein fraction (approx. 27g) containing other proteins was recovered from the ion exchange column by elution with 1M sodium chloride in 20 mM sodium citrate pH 3. The BBI containing fraction yielded a surprisingly and unexpectedly pure BBI composition. It was expected that this fraction would contain additional proteins (including, for example, KTI and other soy whey proteins with a pI at or below that of BBI).

The BBI protein stream (approx. 6.45 I) was brought to 40% saturation with (NH₄)₂SO₄ for approximately 30 minutes and at a temperature of approximately 23° C. to form a supernatant and a precipitated BBI protein fraction, which were separated via centrifugation.

The precipitated BBI protein fraction was contacted twice with a 45% saturated (NH₄)₂SO₄ washing medium for approximately 5 minutes each and at a temperature of approximately 23° C.

The precipitated BBI protein fraction was solubilized in a minimal volume of deionized water and transferred to Pierce 2K molecular weight cutoff Slide-a-lyzer dialysis cassettes and dialyzed extensively against deionized water. The BBI protein fraction was recovered from the dialysis cassettes and centrifuged to remove a small amount of precipitated material.

The soluble BBI protein fraction was brought to a temperature of 4° C. and sufficient 10% Triton X114 solution (at a temperature of 4° C.) was added to yield a final Triton X114 concentration of 1%. This mixture was stirred for approximately 60 hours at a temperature of 4° C. The mixture was heated to approximately 40° C. for 30 minutes to bring about cloud point precipitation (phase separation) of Triton X114.

The mixture was centrifuged and the upper BBI protein fraction phase was collected. The endotoxin-enriched lower Triton X114 phase was contacted with a deionized water washing medium at a temperature of 4° C. for 30 minutes. The mixture was heated to a temperature of 40° C. for 30 minutes to bring about cloud point precipitation (phase separation) of Triton X114. The mixture was centrifuged and the upper residual BBI protein fraction phase was collected and combined with the previous BBI protein fraction material. Surprisingly and unexpectedly, Triton X114 solution performed much better than other solutions to remove endotoxin.

The total BBI protein fraction was passed through Pall Life Sciences 0.2 microns HT Tuffryn membrane Acrodisc syringe filters into ethanol-rinsed glass vials. The vials were frozen at −80° C. and lyophilized on a Labconco Freezone 4.5 freeze dry system.

Approximately 2.9 g of BBI (>95% purity as determined by SDS PAGE) were recovered. Table 1 illustrates the results obtained at each purification step in accordance with the process described in Example 1, ultimately achieving a high purity product.

TABLE 1

| | | | | | | Spec | | | |
|---|---|---|---|---|---|---|---|---|---|
| | | | Total | % | Ci | activity (Ci | | % | |
| | Volume | [protein] | protein | Recovery | Activity | units/gm | Total Ci | Recovery | Fold |
| Step | (L) | (mg/ml) | (gm) | Protein | (Units/L) | protein) | Units | Activity | purity |
| Soy Whey | 37.41 | 8.3 | 310.503 | | 952 | 114.7 | 35615 | | |
| Microfiltration | 34.056 | 6.7 | 228.1752 | 73.5 | 801 | 119.6 | 27290 | 76.6 | 1.0 |
| Ultrafiltration | 0.516 | 210.8 | 108.7728 | 35.0 | 43509 | 206 | 22451 | 63.0 | 1.8 |
| CE | 6.45 | 1.52 | 9.804 | 3.2 | 3069 | 2019 | 19794 | 55.6 | 17.6 |
| Chromatography | | | | | | | | | |
| Ammonium | 0.2 | 16.2 | 3.24 | 1.0 | 35932 | 2218 | 7186 | 20.2 | 19.3 |
| Sulfate pptn | | | | | | | | | |
| Endotoxin | 0.22 | 13.2 | 2.904 | 0.9 | 31020 | 2350 | 6824 | 19.2 | 20.5 |
| removal | | | | | | | | | |

### Example 2

### Recovery of BBI protein from soy whey protein

Spray-dried soy whey protein (44 gm) having a total protein content of 86.2 wt. % (dry wt. basis as determined by nitrogen combustion assay, standard Kjeldahl method) was resuspended to a final concentration of 10% (w/v) in deionized water, and stirred for 2 hours at room temperature. Suspension was then centrifuged at 4000×G in a Beckman JA-10 rotor for 10 minutes to remove insolubles. The supernatant was diluted with 4 volumes of Sodium Citrate buffer, 10 mM, pH 3.0, and further adjusted to a final pH of 3.0 with concentrated hydrochloric acid. The pH-adjusted supernatant was centrifuged to remove insolubles at 4000 RPM in a Jouan C60 rotor for 30 minutes at room temperature, and the supernatant decanted and used as the column load.

Solutions used for column development were as follows: Solution A: Deionized water; Solution B: Sodium citrate, 500 mM, pH 2.1. A 21.6×5 cm column of SP Sepharose Fast Flow resin (424 ml) in a Axichrom 50/300 column (GE Healthcare, Piscataway, N.J.) was equilibrated in 3 column volumes of 98% Solution A, 2% Solution B. The final soy whey protein solution described in the previous paragraph (2.29 liters, 14.2 mg/ml protein estimated using a modified Lowry procedure, Sigma-Aldrich Total Protein Kit, Micro-Lowry, Onishi and Barr Modification) was applied to the column at a flow rate of 50 ml/min. The column was washed with 98% Solution A, 2% Solution B (20 column volumes), then eluted with a linear 2-15% gradient of Solution B over 5 column volumes, followed by isocratic 15% Solution B for an additional 20 column volumes. Elution was then performed at isocratic 20% Solution B for an additional 20 column volumes, followed by 100% Solution B for 5 column volumes.

Representative fractions were collected throughout the column elution phase. Fraction 1 (1740 ml) was collected from 297 to 2120 ml. The entire 15% isocratic elution step was collected as fraction 2 (8500 ml). The entire 20% Solution β isocratic step was collected as fraction 3 (8500 ml). Fraction 4 (2120 ml) comprised the 100% Solution B elution. Purified BBI was identified following SDS-PAGE analysis on 10-20% Criterion Tris-HCl gels (Bio-Rad Labs, Hercules, Calif.) in fraction 2.

The soluble BBI protein fraction was brought to a temperature of 4° C. and sufficient 10% Triton X114 solution (at a temperature of 4° C.) was added to yield a final Triton X114 concentration of 1%. This mixture was stirred for approximately 60 hours at a temperature of 4° C. The mixture was heated to approximately 40° C. for 30 minutes to bring about cloud point precipitation (phase separation) of Triton X114.

The mixture was centrifuged and the upper BBI protein fraction phase was collected. The endotoxin-enriched lower Triton X114 phase was contacted with a deionized water washing medium at a temperature of 4° C. for 30 minutes. The mixture was heated to a temperature of 40° C. for 30 minutes to bring about cloud point precipitation (phase separation) of Triton X114. The mixture was centrifuged and the upper residual BBI protein fraction phase was collected and combined with the previous BBI protein fraction material. This material was then partially lyophilized using a Virtis Freezemobile 25XL to reduce the volume of sample to approximately 150 ml.

The total BBI protein fraction was passed through Pall Life Sciences 0.2 microns HT Tuffryn membrane Acrodisc syringe filters into ethanol-rinsed glass vials. The vials were frozen at −80° C. and lyophilized on a Virtis Freezemobile 25XL freeze dry system.

Approximately 3.6 g of BBI (>95% purity as determined by SDS-PAGE) were recovered. Table 2 illustrates the results obtained at each purification step in accordance with the process described in Example 2, ultimately achieving a high purity product.

TABLE 2

| | | | | | | Spec | | | |
|---|---|---|---|---|---|---|---|---|---|
| | | | Total | % | Ci | activity (Ci | Total | % | |
| | Volume | [protein] | protein | Recovery | Activity | units/gm | Ci | Recovery | Fold |
| Step | (L) | (mg/ml) | (gm) | Protein | (Units/L) | protein | Units | Activity | purity |
| Soy Whey Protein | 2.29 | 14.2 | 32.5 | 100 | 4019 | 283 | 9203 | 100 | 1 |
| CE | 8.5 | 0.565 | 4.8 | 14.8 | 919 | 1627 | 7811 | 85 | 5.7 |
| Chromatography | | | | | | | | | |
| Diafiltration | 0.7 | 6.15 | 4.3 | 13.2 | 10320 | 1678 | 7224 | 78 | 5.9 |
| Endotoxin Removal | 1.05 | 4.49 | 4.7 | 14.5 | 7575 | 1687 | 7953 | 86 | 6.0 |

### Example 3

### Comparison of BBI Protein Sample to BBI Protein of Present Invention

As a comparison between the known BBI protein structures and the BBI protein of the present invention, Table 3 sets forth the mole percent (mol %) of the amino acid residues found in each. The BBI product of the present invention was analyzed by Molecular Structure Facility at UC Davis using an L-8800 Hitachi analyzer. The analyzer used ion-exchange chromatography to separate amino acids followed by a “post-column” ninhydrin reaction detection system. The standard hydrolysis procedure used 6N HCl for 24 hours at 110° C. Cysteine (and cystine) and methionine were determined by oxidation with performic acid, which yielded the acid stable forms of cysteic acid and methionine sulfone, prior to the standard acid hydrolysis. Tryptophan was determined using a MES hydrolysis step.

TABLE 3
Comparison of Amino Acid Residues found in Known BBI
Protein and BBI Protein Isoform (E113609-146).

| | Theoretical | BBIS (E113609-146) | |
|---|---|---|---|
| Amino Acid | BBI mole % | Measured mole % | Difference |
| Asx | 15.5 | 15.3 | −0.2 |
| Thr | 2.8 | 3.3 | 0.5 |
| Ser | 12.7 | 12.0 | −0.7 |
| Glx | 9.8 | 9.0 | −0.8 |
| Pro | 8.5 | 8.4 | −0.1 |
| Gly | 0.0 | 1.2 | 1.2 |
| Ala | 5.6 | 5.7 | 0.1 |
| Val | 1.4 | 1.5 | 0.1 |
| Ile | 2.8 | 2.8 | 0.0 |
| Leu | 2.8 | 3.7 | 0.9 |
| Tyr | 2.8 | 2.8 | 0.0 |
| Phe | 2.8 | 2.9 | 0.1 |
| His | 1.4 | 1.5 | 0.1 |
| Lys | 7.0 | 6.4 | −0.6 |
| Arg | 2.8 | 3.3 | 0.5 |
| Cys | 19.7 | 18.4 | −1.3 |
| Met | 1.4 | 1.6 | 0.2 |
| Trp | 0.0 | 0.0 | 0.0 |

### Example 4

### BBI for Treating a Pathological State

As discussed previously, a BBI protein of the invention is used to treat certain pathological states in a subject. A BBI protein is a single BBI protein or any mixture of BBI proteins described herein, A BBI protein of the invention can be administered as, for example, a composition comprising a BBI protein of the invention and pharmaceutically acceptable carrier or as a food comprising a BBI protein of the invention.

It is found that a BBI protein of the invention prevents loss of functional skeletal muscle mass and force during periods of non-use. Addition of a BBI protein of the invention to the diet is found to significantly attenuate skeletal muscle atrophy following periods of hindlimb suspension. Further, administration of a BBI protein of the invention is found to produce functional improvement of dystrophic muscles in mdx mice, thus demonstrating further use of compositions comprising a BBI protein of the invention for treatment of degenerative muscle disorders including, for example, muscular dystrophy, amyotrophic lateral sclerosis, spinal muscle atrophy and spinal cord injury.

Hindlimb suspension experiment and methods are described previously and known to those of ordinary skill in the art (see, for example, Matuszczak et al., Aviat Space Environ Med 75: 581-588, 2004; Arbogast et al., Journal of Applied Physiology March 2007 vol. 102 no. 3: 956-964; U.S. Pre-Grant Publication No. 20080300179). Neurodegenerative muscle disorder experiments and methods are described previously and known to those of ordinary skill in the art (see, for example, Morris et al., J Appl Physiol. 2010 November; 109(5):1492-9; U.S. Pre-Grant Publication No. 20080300179).

The ability of a composition comprising a BBI protein of the invention to inhibit the progression of muscle atrophy associated with non-use is demonstrated in mice by measurement of a number of physiological parameters known to change during muscle unloading (e.g., hindlimb suspension). Results obtained in BBI treated (i.e., administration of a composition comprising a BBI protein of the invention) suspended and non-suspended animals is compared against those fed either aBBI (i.e., administration of a composition comprising a BBI protein of the invention that is autoclaved to remove inhibitory activity) or standard chow. For each experiment, mice fed one of the three types of feed is subjected to hindlimb suspension or use as non-suspended controls.

In initial experiments, three-month-old mice are used to demonstrate the ability of BBI-supplemented food to reduce the amount of muscle atrophy associated with hindlimb suspension. For this experiment, mice suspended for 14 days are given either BBI- or aBBI-supplemented food. Following suspension, the muscles are dissected and force measured. The tetanic force is higher in the BBI-fed animals than in aBBI-fed animals. The mean specific force, measured in tension per gram muscle weight is greater in the BBI-fed animals than in the aBBI-fed animals. The muscle weight of the BBI-fed animals is greater than the muscle weight of the aBBI-fed animals. The percent atrophy the aBBI-fed animals is greater than the BBI-fed animals.

As an increase in muscle weight is observed in the BBI-fed animals, a larger study size using six-month-old mice is performed.

In these experiments, body weights of suspended and non-suspended mice is measured prior to and following the experimental period. Non-suspended animals in each group exhibit slight increases in body weight over 14 days. Body weight of suspended BBI-fed and aBBI-fed animals over 14 days of hindlimb suspension is decreased. Body weight of suspended control-fed animals over 14 days of hindlimb suspension is decreased. Body mass decline has been reported previously by many studies (see refs. in Thomason, D. B. and Booth, F. W. J Appl Physiol 1990 68:1-12) and has been suggested to be due to both a reduction in total food intake and a reduction in weight gain per gram of food eaten (Morey E R. Bioscience 29: 168-172, 1979).

To determine whether a BBI protein of the invention is able to attenuate muscle loss during non-use atrophy, animals fed either control food or food supplemented with BBI are suspended for 3, 7, or 14 days. Dietary supplementation with BBI is found to attenuate the loss of muscle mass at each time point. After 7 days of hindlimb suspension, the muscle mass of the BBI-fed animals is greater than aBBI-fed and control-fed animals. The average soleus muscle weight of the BBI-fed animals is greater than aBBI-fed and control-fed animals. The percent atrophy of the BBI-fed animals is limited and decreased when compared to aBBI-fed and control-fed animals. The muscle weight of control-fed non-suspended, BBI-fed non-suspended and aBBI-fed non-suspended is not different.

Average fiber number per muscle is similar for all groups suggesting that hindlimb suspension does not induce elimination of individual muscle fibers. Thus, the fiber area of the individual muscle fibers is measured in cross-sections. A simple method to determine whether there is any change in fiber size is to quantify the number of fibers in a high-powered field (e.g. 40×objective). Increased fiber size reduces the number of fibers in the field of view (i.e. the smaller the muscle fibers, the greater the fiber number). Using this method, the average fiber number for the BBI-fed animals in the hindlimb studies is lower compared to aBBI-fed animals, which demonstrates a decrease in atrophy in the BBI-fed animals.

The laminin-stained muscle cross-sections are analyzed to directly measure the fiber area. The mean fiber area is increased in BBI-fed animals when compared to aBBI-fed aminals.

Thus, administration of a BBI protein of the invention ameliorates muscle atrophy associated with hindlimb suspension by at least slowing the decrease in fiber size, thereby maintaining the overall mass of the muscle.

To determine whether the muscle remained functional, contractile measurements on the soleus muscle of both the non-suspended and suspended animals is performed in all the feed groups. The total tetanic force produced by the BBI-fed suspended animals is greater than similarly control-fed and aBBI-fed animals. The results demonstrate a BBI protein of the invention can maintain functional muscle mass and enabling overall greater force production by the muscle in a model of muscle atrophy.

Changes in muscle mass observed in the mice are correlated with BBI intake. More specifically, quantity of food consumed over the 14-day experimental period is plotted against the muscle weights of the individual animals. The results are indicative of a positive correlation between the amount of BBI food consumed per day and muscle weight. The effect of BBI on muscle weight as a function of food intake per day is increased in comparison to aBBI intake. Re-evaluation of the BBI-fed animals to the subset that consumed greater amounts of BBI, show a further reduction in the amount of muscle atrophy when evaluating soleus muscle. Similar analysis in the aBBI-fed mice indicate no such change of soleus muscle. This indicates an increase in consumption of BBI reduces the degree of muscle atrophy (i.e., a dose response). These results indicate that the quantity of food, or more specifically the quantity of BBI, consumed is important in reducing the amount of muscle atrophy associated with hindlimb suspension.

In additional experiments, osmotic pumps are inserted to directly deliver either a BBI protein of the invention or aBBI protein of the invention to six month old mice. Each animal has an Alzet osmotic pump (Alza, Palo Alto, Calif.) containing either BBI (10% w/v) or aBBI (10% w/v) surgically inserted on the anterior portion of the back, directly under the skin. The pumps release the solution constantly over a period of two weeks at a rate of, for example, 0.5 .mu.l/hr. The muscle weight of the BBI treated animals is greater than the muscle weight of the aBBI treated animals. The maintenance of muscle mass by BBI results in an enhancement in muscle weight following 14 days suspension. Experiments are also performed in mdx mice, a murine model for Duchenne muscular dystrophy.

In these experiments, treatment of male mdx mice with a composition comprising a BBI protein of the invention, specifically food supplemented therewith, is initiated at four weeks of age and continued for 12 weeks. The weights of the animals are monitored and recorded each week. No difference in body weight increases between the control mdx mice and those provided food supplemented with 1.0% BBI are observed. In addition, as a further control, wild type C57BL/6 mice are provided food supplemented with BBI to determine whether BBI induces any changes in normal, non-dystrophic muscle size or function.

The diaphragm of mdx mice exhibits considerable fibrosis at 4 months of age that is observable using routine hematoxylin-eosin (H&E) staining. Greater differentiation of fibrotic tissue from the muscle cells can be achieved using a trichrome method which stains muscle tissue red and stains fibrotic and connective tissue dark blue. Feeding with BBI is found to markedly improve the appearance of the diaphragms of mdx mice stained using H&E and trichrome as compared to control mdx mice.

Further, the muscle fibers of the mdx mice undergo pronounced cycles of degeneration/regeneration beginning at approximately 4 weeks of age. Regeneration of muscle fibers requires activation and fusion of satellite cells that appear in the center of the regenerating fibers. Thus, a measure of regenerating muscle fibers is the presence of central nucleated muscle fibers (CNF) with an increased proportion of CNFs representing increased regeneration. Muscle sections are stained with laminin to outline the muscle fibers and the nuclei are stained with the nuclear stain 4,6-diamidino-2-phenylindole. For each muscle, the number of CNFs is determined as a proportion of the total fiber number with a total of 2-4 muscles used for each measurement. A significant reduction in the proportion of CNFs in the tibialis anterior muscles, EDL muscles, and diaphragm muscles is observed following BBI treatment.

Evan's blue dye is used to determine the membrane integrity of both untreated and BBI treated mdx mice. Twenty-four hours prior to sacrifice, animals are intra-peritoneally injected with Evan's Blue dye. The muscles are sectioned, fixed, and observed under a fluorescent microscope to determine the degree of membrane damage. Increased regions of infiltration are observed in the quadricep muscles of at least one untreated mdx animal.

EDL muscles of mdx mice demonstrate an increase in mass and cross-sectional area in comparison to non-dystrophic animals. However, not all increases in mass correlate to improvement in the force per cross-sectional area (specific force), rather there can be a significant decline in the specific force of mdx muscles. BBI treatment significantly increases muscle mass, absolute force, and cross-sectional area, while maintaining specific force. These results indicate strength improvement is gained by BBI treatment. Though the specific force is unchanged, the increased muscle mass and absolute force provides the animal with a greater ability to perform everyday tasks. The increased muscle mass is not simply due to an overall increase in body weight as there is a significant increase in the muscle weight to body weight ratio.

Thus, as demonstrated by each of the above-described examples, there is significant improvement in multiple morphological and functional measurements of skeletal muscle following twelve weeks of BBI consumption by mice of this murine model for Duchenne muscular dystrophy.

Accordingly, the present invention provides methods for use of a composition comprising a BBI protein of the invention

As also demonstrated herein, administration of a composition comprising a BBI protein of the invention improves skeletal muscle function, resulting from both an increase strength and increased mass of the muscle in a murine model for a degenerative skeletal muscle disorder.

Further, the present invention provides compositions and methods for alleviating symptoms and/or slowing of progression of degenerative skeletal muscle diseases or disorders. As demonstrated herein, treatment with a composition comprising a BBI protein of the invention improves skeletal muscle function in a murine model for the degenerative skeletal muscle disorder Duchenne muscular dystrophy.

One skilled in the art would readily appreciate that the methods and compositions described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims,

What is claimed is:

1. A Bowman-Birk protease inhibitor (BBI) product having a total BBI protein concentration of at least about 90 wt. %.

2. The BBI product of claim 1, wherein the BBI product has a total BBI protein concentration of at least about 95 wt. %.

3. The BBI product of claim 1, wherein the BBI product has a total BBI protein concentration of at least about 99 wt. %.

4. The BBI product of claim 1, wherein the BBI protein comprises at least one amino acid sequence having at least an 85% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

5. The BBI product of claim 1, wherein the BBI protein comprises at least one amino acid sequence having at least a 90% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

6. The BBI product of claim 1, wherein the BBI protein comprises at least one amino acid sequence having at least a 95% identical to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

7. The BBI product of claim 1, wherein the product further exhibits a trypsin inhibitor activity of at least about 1200 trypsin inhibition units/g protein.

8. The BBI product of claim 1, wherein the product further exhibits a chymotrypsin inhibitor activity of at least about 1600 chymotrypsin inhibition units/g protein.

9. The BBI product of claim 1, wherein the product further comprises a total endotoxin content of no more than about 5 endotoxin units/g protein.

10. A method of treating a pathological state, wherein the method comprises administering to a subject an effective amount of a BBI product of claim 1.

11. The method of claim 10, wherein the pathological state is a disease associated with muscle dysfunction, a cell proliferative disorder, an autoimmune disease, or a skin disorder.

12. A method of promoting skin health, wherein the method comprises administering to a subject an effective amount of a BBI product of claim 1.

13. A Bowman-Birk protease inhibitor (BBI) product wherein the BBI protein comprises at least one amino acid sequence having at least an 80% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and any combination thereof.

14. The BBI product of claim 13, wherein the BBI protein comprises at least one amino acid sequence having at least an 85% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

15. The BBI product of claim 13, wherein the BBI protein comprises at least one amino acid sequence having at least a 90% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

16. The BBI product of claim 13, wherein the BBI protein comprises at least one amino acid sequence having at feast a 95% identical to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

17. A Bowman-Birk protease inhibitor (BBI) product having a total BBI protein concentration of at least about 90 wt. % and wherein the BBI protein comprises at least one amino acid sequence having at least an 80% indentity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, and combinations thereof.

18. A Bowman-Birk protease inhibitor (BBI) product having a total protein content of at least about 95% on a dry weight basis, a trypsin inhibitor activity of at least about 1200 trypsin inhibition units/g protein, and a chymotrypsin inhibitor activity of at least about 1600 chymotrypsin inhibition units/g protein.

19. The BBI product of claim 18, further comprising a total endotoxin content of no more than about 5 endotoxin units/g protein.

20. A Bowman-Birk protease inhibitor (BBI) product having a total BBI protein concentration of at least about 90 wt. % BBI protein, wherein the BBI protein consists of at least one amino acid sequence having at least an 80% identity to SEQ ID NO: 1, SEQ ID NO: 2, SEQ ID NO: 3, SEQ ID NO: 4, SEQ ID NO: 5, SEQ ID NO: 6, and any combination thereof, and further wherein the BBI product exhibits one or more of the following properties:
(i) a trypsin inhibitor activity of at least about 1200 trypsin inhibition units/g protein;
(ii) a chymotrypsin inhibitor activity of at least about 1600 chymotrypsin inhibition units/g protein; and/or
(iii) a total endotoxin content of no more than about 5 endotoxin units/g protein.


SEQUENCE LISTING (6 sequences)

SEQ ID NO: 1 - protein, 71 aa, Soybean
DDESSKPCCDQCACTKSNPPQCRCSDMRLNSCHSACKSCICALSYPAQCFCVDITDFCYEPCKPSEDDKEN

SEQ ID NO: 2 - protein, 70 aa, Soybean
DDESSKPCCDQCACTKSNPPQCRCSDMRLNSCHSACKSCICALSYPAQCFCVDITDFCYEPCKPSEDDKE

SEQ ID NO: 3 - protein, 69 aa, Soybean
DDESSKPCCDQCACTKSNPPQCRCSDMRLNSCHSACKSCICALSYPAQCFCVDITDFCYEPCKPSEDDK

SEQ ID NO: 4 - protein, 68 aa, Soybean
DDESSKPCCDQCACTKSNPPQCRCSDMRLNSCHSACKSCICALSYPAQCFCVDITDFCYEPCKPSEDD

SEQ ID NO: 5 - protein, 67 aa, Soybean
DDESSKPCCDQCACTKSNPPQCRCSDMRLNSCHSACKSCICALSYPAQCFCVDITDFCYEPCKPSED

SEQ ID NO: 6 - protein, 66 aa, Soybean
DDESSKPCCDQCACTKSNPPQCRCSDMRLNSCHSACKSCICALSYPAQCFCVDITDFCYEPCKPSE